SEQUENCE LISTING (16 sequences)

SEQ ID NO: 1 - DNA, 7009 bp, synthetic construct
  misc_feature 1..7009: note = pTR22-smCBA-otoferlinNT-APSD-APhead
  source 1..7009: mol_type = other DNA
aggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagcaaaggcagagagaagaccaagggaggcagagatggcgagcacaaagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgactgttaattaagcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgc

SEQ ID NO: 2 - DNA, 7413 bp, synthetic construct
  misc_feature 1..7413: note = pTR22-APhead-APSA-otoferlinCT
  source 1..7413: mol_type = other DNA
aggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctggcgcgcccaattggcttcgaattctagcggccgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaaccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctgagcggccgcggtaccaagggcgaattctgcagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggactagtccgtcgactgttaattaagcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgc

SEQ ID NO: 3 - DNA, 287 bp, synthetic construct
  misc_feature 1..287: note = Synthetic polynucleotide
  source 1..287: mol_type = other DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttc

SEQ ID NO: 4 - DNA, 650 bp, synthetic construct
  misc_feature 1..650: note = Synthetic polynucleotide
  source 1..650: mol_type = other DNA
ggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 5 - protein, 1997 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKLLLLLLLLLLLALFLYSVPGYLVKKILGA

SEQ ID NO: 6 - protein, 1997 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLGLFLYSLPGYMVKKLLGA

SEQ ID NO: 7 - DNA, 84 bp, synthetic construct
  misc_feature 1..84: note = Synthetic polynucleotide
  source 1..84: mol_type = other DNA
gtaagtatcaaggttacaagacaggtttaaggagaccaatagaaactgggcttgtcgagacagagaagactcttgcgtttctga

SEQ ID NO: 8 - DNA, 49 bp, synthetic construct
  misc_feature 1..49: note = Synthetic polynucleotide
  source 1..49: mol_type = other DNA
taggcacctattggtcttactgacatccactttgcctttctctccacag

SEQ ID NO: 9 - protein, 1992 aa, Mus musculus
MALIVHLKTVSELRGKGDRIAKVTFRGQSFYSRVLENCEGVADFDETFRWPVASSIDRNEVLEIQIFNYSKVFSNKLIGTFCMVLQKVVEENRVEVTDTLMDDSNAIIKTSLSMEVRYQATDGTVGPWDDGDFLGDESLQEEKDSQETDGLLPGSRPSTRISGEKSFRSKGREKTKGGRDGEHKAGRSVFSAMKLGKTRSHKEEPQRQDEPAVLEMEDLDHLAIQLGDGLDPDSVSLASVTALTSNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISAGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLMLGLAVEILDTSNPELTSSTEVQVEQATPVSESCTGRMEEFFLFGAFLEASMIDRKNGDKPITFEVTIGNYGNEVDGMSRPLRPRPRKEPGDEEEVDLIQNSSDDEGDEAGDLASVSSTPPMRPQITDRNYFHLPYLERKPCIYIKSWWPDQRRRLYNANIMDHIADKLEEGLNDVQEMIKTEKSYPERRLRGVLEELSCGCHRFLSLSDKDQGRSSRTRLDRERLKSCMRELESMGQQAKSLRAQVKRHTVRDKLRSCQNFLQKLRFLADEPQHSIPDVFIWMMSNNKRIAYARVPSKDLLFSIVEEELGKDCAKVKTLFLKLPGKRGFGSAGWTVQAKLELYLWLGLSKQRKDFLCGLPCGFEEVKAAQGLGLHSFPPISLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGSATAGDLLAAFELLQIGPSGKADLPPINGPVDMDRGPIMPVPVGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPNWNTTGEVVVSMEPEEPVKKLETMVKLDATSDAVVKVDVAEDEKERKKKKKKGPSEEPEEEEPDESMLDWWSKYFASIDTMKEQLRQHETSGTDLEEKEEMESAEGLKGPMKSKEKSRAAKEEKKKKNQSPGPGQGSEAPEKKKAKIDELKVYPKELESEFDSFEDWLHTFNLLRGKTGDDEDGSTEEERIVGRFKGSLCVYKVPLPEDVSREAGYDPTYGMFQGIPSNDPINVLVRIYVVRATDLHPADINGKADPYIAIKLGKTDIRDKENYISKQLNPVFGKSFDIEASFPMESMLTVAVYDWDLVGTDDLIGETKIDLENRFYSKHRATCGIAQTYSIHGYNIWRDPMKPSQILTRLCKEGKVDGPHFGPHGRVRVANRVFTGPSEIEDENGQRKPTDEHVALSALRHWEDIPRVGCRLVPEHVETRPLLNPDKPGIEQGRLELWVDMFPMDMPAPGTPLDISPRKPKKYELRVIVWNTDEVVLEDDDFFTGEKSSDIFVRGWLKGQQEDKQDTDVHYHSLTGEGNFNWRYLFPFDYLAAEEKIVMSKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLALFLYSLPGYMVKKLLGA

SEQ ID NO: 10 - DNA, 143 bp, synthetic construct
  misc_feature 1..143: note = Synthetic polynucleotide
  source 1..143: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttc

SEQ ID NO: 11 - DNA, 143 bp, synthetic construct
  misc_feature 1..143: note = Synthetic polynucleotide
  source 1..143: mol_type = other DNA
acccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacc

SEQ ID NO: 12 - DNA, 21 bp, synthetic construct
  misc_feature 1..21: note = Synthetic polynucleotide
  source 1..21: mol_type = other DNA
cacttgctttgtctcatctcc

SEQ ID NO: 13 - DNA, 21 bp, synthetic construct
  misc_feature 1..21: note = Synthetic polynucleotide
  source 1..21: mol_type = other DNA
gtcacttcttctgggtatttc

SEQ ID NO: 14 - DNA, 6967 bp, synthetic construct
  misc_feature 1..6967: note = pTR22-smCBA-otoferlinNT Hs var 1 5-APSD-APhead
  source 1..6967: mol_type = other DNA
aggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgactgttaattaagcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgc

SEQ ID NO: 15 - DNA, 7470 bp, synthetic construct
  misc_feature 1..7470: note = pTR22-APhead-APSA-otoferlinCT Hs var 1
  source 1..7470: mol_type = other DNA
aggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctggcgcgcccaattggcttcgaattctagcggccgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacgagcttcatctggttcctgaaccctctcaagtcggctcgctacttcttgtggcacacgtatcgctggctgctcctcaaactgttgctgctcctgctgctgctcctcctcctcgccctgttcctctactctgtgcctggctacctggtcaagaaaatcctcggggcctgagcggccgcggtaccaagggcgaattctgcagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggactagtccgtcgactgttaattaagcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgc

SEQ ID NO: 16 - DNA, 7470 bp, synthetic construct
  misc_feature 1..7470: note = pTR22-APhead-APSA-otoferlinCT Hs var 5
  source 1..7470: mol_type = other DNA
aggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctggcgcgcccaattggcttcgaattctagcggccgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgagcggccgcggtaccaagggcgaattctgcagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggactagtccgtcgactgttaattaagcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgc

(19) United States
(12) Patent Application Publication
(10) Pub. No.: US 2025/0367325 A1
(43) Pub. Date: Dec. 4, 2025

(54) COMPOSITIONS AND METHODS FOR EXPRESSING OTOFERLIN

(71) Applicants: University of Florida Research Foundation, Incorporated, Gainesville, FL (US); The Regents of the University of California, Oakland, CA (US)

(72) Inventors: Sanford L. Boye, Gainesville, FL (US); Frank M. Dyka, Gainesville, FL (US); William W. Hauswirth, Gainesville, FL (US); Omar Akil, San Francisco, CA (US)

(73) Assignees: University of Florida Research Foundation, Incorporated, Gainesville, FL (US); The Regents of the University of California, Oakland, CA (US)

(21) Appl. No.: 19/233,471

(22) Filed: Jun. 10, 2025

Related U.S. Application Data
(63) Continuation of application No. 16/611,049, filed on Nov. 5, 2019, now Pat. No. 12,359,221.
(60) Provisional application No. 62/502,462, filed on May 5, 2017.

(51) Int. Cl. A61K 48/00; A61K 38/17; C07K 14/435; C12N 15/86

(52) U.S. Cl. CPC A61K 48/0058; A61K 38/1709; C07K 14/435; C12N 15/86; C12N 2750/14143; C12N 2800/40; C12N 2840/445

(57) ABSTRACT

Provided herein are methods and compositions for expressing Otoferlin, e.g., utilizing adeno-associated viral (AAV) particles. Such methods and compositions may be useful for treatment of diseases such as Deafness, Autosomal Recessive 9 (DFNB9).

### CROSS-REFERENCE TO RELATED APPLICATIONS

This application claims priority to U.S. Provisional Application No. 62/502,462 filed on May 5, 2017, the entire disclosure of which is incorporated by reference herein.

### FEDERALLY SPONSORED RESEARCH

This invention was made with government support under grants EY000331, EY021721 and DC012118 awarded by the National Institutes of Health. The government has certain rights in the invention.

### BACKGROUND OF INVENTION

Nonsyndromic deafness is a form of hearing loss that is generally caused by defects or damage to the inner ear and/or middle ear. Mutations in the OTOF gene, which encodes the protein Otoferlin, are thought to cause a type of nonsyndromic deafness called Deafness, Autosomal Recessive 9 (DFNB9). Treatment of DFNB9 and other similar forms of deafness currently involves using cochlear implants for severe or profound hearing loss and hearing aids for milder forms of hearing loss. There remains a need for alternative treatment forms that do not rely on or rely less heavily on electronic devices for restoring hearing.

### SUMMARY OF INVENTION

Provided herein are compositions and methods for expressing Otoferlin, e.g., in a cell or subject. As described herein, it has been found that delivery of the OTOF cDNA to otof knock-out mice via a dual adeno-associated virus (AAV) system containing different portions of the OTOF cDNA was capable of rescuing hearing in the mice to near wild-type levels.

In some aspects, the disclosure provides a method of increasing expression of Otoferlin in a cell, the method comprising contacting the cell with a first AAV particle comprising a first polynucleotide; and contacting the cell with a second AAV particle comprising a second polynucleotide, wherein the first polynucleotide comprises inverted terminal repeat sequences flanking an expression cassette containing, from 5′ to 3′: (a) a promoter, (b) a partial coding sequence that encodes an N-terminal portion of an Otoferlin polypeptide, (c) a splice donor site, and (d) a first region of homology containing a sequence that is homologous to a sequence in the second polynucleotide, and the second polynucleotide comprises inverted terminal repeat sequences flanking an expression cassette containing, from 5′ to 3′: (a) a second region of homology containing a sequence that is homologous to a sequence in the first polynucleotide, (b) a splice acceptor site, (c) a partial coding sequence that encodes a C-terminal portion of the Otoferlin polypeptide, and (d) a polyadenylation (pA) signal sequence.

In some embodiments, the region of homology in the first and second polynucleotides is between 50 and 500 nucleotides. In some embodiments, the region of homology in the first and second polynucleotides is between 50 and 300 nucleotides. In some embodiments, the region of homology comprises the nucleotide sequence of SEQ ID NO: 3. In some embodiments, the promoter is a chimeric CMV β actin (smcBA) promoter. In some embodiments, the promoter comprises the sequence of SEQ ID NO: 4. In some embodiments, the Otoferlin polypeptide comprises the amino acid sequence of SEQ ID NO: 5 or SEQ ID NO: 6. In some embodiments, the splice donor site comprises the sequence of SEQ ID NO: 7. In some embodiments, the splice acceptor site comprises the sequence of SEQ ID NO: 8. In some embodiments, the inverted terminal repeat sequences are AAV2 inverted terminal repeat sequences. In some embodiments, the first and second AAV particle are AAV2 serotype particles. In some embodiments, the cell is ex vivo. In some embodiments, the cell is in vivo. In some embodiments, the cell is in a mammalian subject. In some embodiments, the subject has Deafness, Autosomal Recessive 9 (DFNB9).

In other aspects, the disclosure provides a composition comprising a first AAV particle comprising a first polynucleotide; and a second AAV particle comprising a second polynucleotide, wherein the first polynucleotide comprises inverted terminal repeat sequences flanking an expression cassette containing, from 5′ to 3′: (a) a promoter, (b) a partial coding sequence that encodes an N-terminal portion of an Otoferlin polypeptide, (c) a splice donor site, and (d) a first region of homology containing a sequence that is homologous to a sequence in the second polynucleotide, and the second polynucleotide comprises inverted terminal repeat sequences flanking an expression cassette containing, from 5′ to 3′: (a) a second region of homology containing a sequence that is homologous to a sequence in the first polynucleotide. (b) a splice acceptor site, (c) a partial coding sequence that encodes a C-terminal portion of the Otoferlin polypeptide, and (d) a polyadenylation (pA) signal sequence.

In some embodiments, the region of homology in the first and second polynucleotides is between 50 and 500 nucleotides. In some embodiments, the region of homology in the first and second polynucleotides is between 50 and 300 nucleotides. In some embodiments, the region of homology comprises the nucleotide sequence of SEQ ID NO: 3. In some embodiments, the promoter is a chimeric CMV ß actin (smcBA) promoter. In some embodiments, the promoter comprises the sequence of SEQ ID NO: 4. In some embodiments, the Otoferlin polypeptide comprises the amino acid sequence of SEQ ID NO: 5 or SEQ ID NO: 6. In some embodiments, the splice donor site comprises the sequence of SEQ ID NO: 7. In some embodiments, the splice acceptor site comprises the sequence of SEQ ID NO: 8. In some embodiments, the inverted terminal repeat sequences are AAV2 inverted terminal repeat sequences. In some embodiments, the first and second AAV particle are AAV2 serotype particles. In some embodiments, the composition further comprises a pharmaceutically acceptable carrier.

In yet other aspects, the disclosure provides a kit comprising a composition as described herein or comprising a first AAV particle as described herein and a second AAV particle as described herein.

These and other aspects are described in more detail herein.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein.

FIG. 1A is a map of a plasmid containing AAV2 inverted terminal repeats (TR) flanking a CMV enhancer, a chicken beta-actin promoter, a 5′ section of the mouse Otoferlin cDNA (Otoferlin NT), a splice donor sequence (APSD), and a homologous sequence for recombination (APhead).

FIG. 1B is a map of a plasmid containing AAV2 inverted terminal repeats (TR) flanking a homologous sequence for recombination (APhead), a splice acceptor sequence (APSA), a 3′ section of the mouse Otoferlin cDNA (Otoferlin CT), a bovine growth hormone polyadenylation signal (bGH PolyA).

FIG. 2 is a schematic of the two expression cassettes in the plasmids in FIGS. 1A and 1B.

FIG. 3A shows the annotated sequence of the expression cassette, including the inverted terminal repeats (TR) for the plasmid in FIG. 1A.

FIG. 3B shows the annotated sequence of the expression cassette, including the inverted terminal repeats (TR) for the plasmid in FIG. 1B.

FIG. 4 is a series of photographs showing expression of OTOF protein in HEK 293 cells treated with AAV2-OTOF-NT (AAV-NT) or AAV2-OTOF-NT and AAV2-OTOF-CT (AAV2-NT+CT).

FIG. 5 is a series of photographs showing expression of GFP in the cochlea organ of Corti surface preparations from wild-type mice treated with AAV2-GFP.

FIGS. 6A-D are a series of photographs and a graph showing OTOF expression in the cochlea of P1-P3 mice. FIG. 6A shows expression of OTOF protein in the mid-turn. FIG. 6B shows expression of OTOF protein in the apex. FIG. 6C shows the difference in OTOF expression in the base, mid-turn and apex in wild-type mice (WT, n=6) and OTOF knock-out mice treated with AAV2-OTOF-NT and AAV2-OTOF-CT (Res. KO NT+CT, n=6). The left bar in each pair of bars is WT and the right bar in each pair of bars is Res. KO NT+CT. FIG. 6D shows RT-PCR data of OTOF mRNA in wild-type (WT), OTOF knock-out mice (KO) and OTOF knock-out mice treated with AAV2-OTOF-NT and AAV2-OTOF-CT (Res. KO).

FIGS. 7A-D show a hearing assessment in mice. FIG. 7A is a trace of auditory brainstem response (ABR) patterns induced by auditory stimuli in wild-type mice (WT), OTOF knock-out mice either untreated (KO/KO NT) or treated (Rescued KO) with AAV2-OTOF-NT and AAV2-OTOF-CT. FIG. 7B shows the auditory brainstem response (ABR) threshold in wild-type mice (WT), untreated Otoferlin knock-out mice (KO), Otoferlin knock-out mice treated with AAV2-OTOF-NT and AAV2-OTOF-CT (Res KO NT+CT), and Otoferlin knock-out mice treated with AAV2-OTOF-NT (KO+NT). FIG. 7C shows a time course of hearing recovery in wild-type mice (WT), untreated OTOF knock-out mice (KO), Otoferlin knock-out mice treated with AAV2-OTOF-NT and AAV2-OTOF-CT (Rescued KO NT+CT), and Otoferlin knock-out mice treated with AAV2-OTOF-NT (KONT). FIG. 7D shows the click ABR threshold in wild-type mice (WT), untreated Otoferlin knock-out mice (KO), Otoferlin knock-out mice treated with AAV2-OTOF-NT and AAV2-OTOF-CT (Res. KO (NT+CT)), and Otoferlin knock-out mice treated with AAV2-OTOF-NT (KO+NT).

FIGS. 8A and B show Otoferlin protein expression in the OTOF rescued KO mice inner hair cells. FIG. 8A shows OTOF protein expression in P12 and older mice treated with AAV2-OTOF-NT and AAV2-OTOF-CT. FIG. 8B shows the percent of inner hair cells expressing OTOF in wild-type mice (WT, n=5) and OTOF knock-out mice treated with AAV2-OTOF-NT and AAV2-OTOF-CT (Rescued KO, n=5). The left bar in each pair of bars is WT and the right bar in each pair of bars is Rescued KO.

FIGS. 9A and 9B are a series of graphs showing hearing assessment. FIG. 9A shows ABR threshold values in wild-type mice (WT), OTOF knock-out mice (KO) and OTOF knock-out mice treated with AAV2-OTOF-NT and AAV2-OTOF-CT (Rescued KO). FIG. 9B shows hearing longevity in WT, KO and Rescued KO mice.

FIG. 10 is a map of a plasmid containing AAV2 inverted terminal repeats (TR) flanking a CMV enhancer, a chicken beta-actin promoter, a 5′ section of a human Otoferlin cDNA (Otoferlin NT), a splice donor sequence (APSD), and a homologous sequence for recombination (APhead).

FIG. 11 is a map of a plasmid containing AAV2 inverted terminal repeats (TR) flanking a homologous sequence for recombination (APhead), a splice acceptor sequence (APSA), a 3′ section of a human Otoferlin cDNA (Otoferlin CT) encoding isoform 1 of Otoferlin, a bovine growth hormone polyadenylation signal (bGH PolyA).

FIG. 12 is a map of a plasmid containing AAV2 inverted terminal repeats (TR) flanking a homologous sequence for recombination (APhead), a splice acceptor sequence (APSA), a 3′ section of the mouse Otoferlin cDNA (Otoferlin CT) encoding isoform 5 of Otoferlin, a bovine growth hormone polyadenylation signal (bGH PolyA).

FIG. 13 shows the annotated sequence of a human OTOF N-terminal expression cassette, including the inverted terminal repeats (TR) for the plasmid in FIG. 10.

FIG. 14 shows the annotated sequence of a human OTOF C-terminal expression cassette for isoform 1, including the inverted terminal repeats (TR) for the plasmid in FIG. 11.

FIG. 15 shows the annotated sequence of a human OTOF C-terminal expression cassette for isoform 5, including the inverted terminal repeats (TR) for the plasmid in FIG. 12.

### DETAILED DESCRIPTION OF INVENTION

As described herein, it has been found that hearing can be restored in Otoferlin knock-out mice by treating the mice with two separate AAV particles, one comprising the 5′ portion of the OTOF cDNA and one comprising the 3′ portion of the OTOF cDNA and each comprising a region of homology for promoting homologous recombination between the 5′ portion and 3′ portion in vivo. This region of homology is flanked by a splice donor sequence on the 5′ side within the 5′ portion of the OTOF cDNA and a splice acceptor sequence on the 3′ side within the 3′ portion of the OTOF cDNA. Accordingly, compositions and methods are provided for increasing expression of Otoferlin.

### Exemplary Definitions

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

As used herein, the terms “nucleic acid” and “polynucleotide sequence” refer to a deoxyribonucleotide or ribonucleotide polymer in either single-or double-stranded form, and unless otherwise limited, encompass known analogs of natural nucleotides that can function in a similar manner as naturally occurring nucleotides.

The term “substantially corresponds to,” “substantially homologous,” or “substantial identity,” as used herein, denote a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides.

When highly-homologous fragments are desired, the extent of percent identity between the two sequences may be at least about 80%, preferably at least about 85%, and more preferably about 90% or 95% or higher, as readily determined by one or more of the sequence comparison algorithms well-known to those of ordinary skill in the art, such as e.g., the FASTA program analysis described by Pearson and Lipman (1988).

### Polynucleotides

In some aspects, polynucleotides are provided for delivering portions of coding sequences of an OTOF gene that encode the Otoferlin protein to a cell. In some embodiments, the coding sequences are derived from a human OTOF gene (see, e.g., NCBI Gene ID: 9381 and cDNA sequences NM_001287489.1, NM_004802.3, NM_194248.2, NM_194322.2, and NM_194323.2). In some embodiments, the coding sequences are derived from a mouse OTOF gene (see, e.g., NCBI Gene ID 83762 and cDNA sequences NM_001100395.1, NM_001286421.1, NM_001313767.1, and NM_031875.2). In some embodiments, a first and a second polynucleotide are provided. It is to be understood that “first,” “second,” “third,” and the like are not meant to imply a particular order or importance unless expressly stated otherwise.

In some embodiments, the first polynucleotide comprises inverted terminal repeat sequences flanking an expression cassette containing, from 5′ to 3′, one or more of (a) a promoter, (b) a partial coding sequence that encodes an N-terminal portion of an Otoferlin polypeptide, (c) a splice donor site, and (d) a first region of homology containing a sequence that is homologous to a sequence in the second polynucleotide. In some embodiments, the first polynucleotide comprises at least two, at least three or all four of (a), (b), (c), and (d).

In some embodiments, the second polynucleotide comprises inverted terminal repeat sequences flanking an expression cassette containing, from 5′ to 3′, one or more of (a) a second region of homology containing a sequence that is homologous to a sequence in the first polynucleotide, (b) a splice acceptor site, (c) a partial coding sequence that encodes a C-terminal portion of the Otoferlin polypeptide, and (d) a polyadenylation (pA) signal sequence. In some embodiments, the second polynucleotide comprises at least two, at least three or all four of (a), (b), (c), and (d).

The partial coding sequences contained within the polynucleotides described herein may be designed so that, upon delivery of the polynucleotides, the partial coding sequences are joined together, e.g., through homologous recombination, and form a complete coding sequence that encodes an Otoferlin polypeptide.

In some embodiments, the polynucleotides are plasmids (e.g., a circular nucleic acid comprising one or more of an origin of replication, a selectable marker, and a reporter gene). In some embodiments, polynucleotides described herein, such as a plasmid, may also contain marker or reporter genes, e.g., LacZ or a fluorescent protein, and an origin of replication. In some embodiments, the plasmid is transfected into a producer cell that produces AAV particles containing the expression cassettes contained within the plasmids.

In some embodiments, the polynucleotides are nucleic acid vectors such as a recombinant adeno-associated virus (AAV) vectors. Exemplary AAV nucleic acid vectors useful according to the disclosure include single-stranded (ss) or self-complementary (sc) AAV nucleic acid vectors.

In some embodiments, recombinant AAV particles comprise the polynucleotides, such as a single-stranded (ss) or self-complementary (sc) AAV nucleic acid vectors. In some embodiments, the polynucleotides contain expression constructs as described herein and inverted terminal repeat (ITR) sequences (e.g., wild-type ITR sequences or engineered ITR sequences) flanking the expression constructs. In some embodiments, the polynucleotides are encapsidated by viral capsids.

Accordingly, in some embodiments, an AAV particle comprises a viral capsid and a polynucleotide as described herein, which is encapsidated by the viral capsid. In some embodiments, the viral capsid comprises 60 capsid protein subunits comprising VP1, VP2 and VP3. In some embodiments, the VP1, VP2, and VP3 subunits are present in the capsid at a ratio of approximately 1:1:10, respectively.

In some embodiments, polynucleotides as described herein (e.g., first and second polynucleotides) comprise regions of homology, e.g., to promote homologous recombination between the polynucleotides once delivered to a cell (see, e.g., Ghosh et al. Efficient transgene reconstitution with hybrid dual AAV vectors carrying the minimized bridging sequences. Hum Gene Ther. 2011 January; 22 (1): 77-83). In some embodiments, a first region of homology and a second region of homology have a threshold level of sequence identity with each other in order to promote homologous recombination. In some embodiments the first region of homology has at least 75%, at least 80%, at least 90%, at least 95%, at least 96%, at least 97%, at least 98%, at least 99% or 100% identity with the second region of homology. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, word-length=12, to obtain sequences with the desired percent sequence identity. To obtain gapped alignments for comparison purposes, Gapped BLAST can be used as described (Altschul et al., 1997). When utilizing BLAST and Gapped BLAST programs, the default parameters of the respective programs (NBLAST and XBLAST) can be used in accordance with published methods. In some embodiments, each region of homology is independently between 50 and 500, 50 and 400, 50 and 300, 100 and 500, 100 and 400, 100 and 300, 200 and 500, 200 and 400, or 200 and 300 nucleotides. In some embodiments, the regions of homology are identical and each region of homology is between 50 and 500, 50 and 400, 50 and 300, 100 and 500, 100 and 400, 100 and 300, 200 and 500, 200 and 400, or 200 and 300 nucleotides. In some embodiments, the region homology comprises a sequence that is at least 75%, at least 80%, at least 85%, at least 90%, at least 95%, at least 96%, at least 97%, at least 98%, at least 99% or 100% identical with the nucleotide sequence

(SEQ ID NO: 3)
CCCCGGGTGCGCGGCGTCGGTGGTGCCGGCGGGGGGCGCCAGGTCGCAG
GCGGTGTAGGGCTCCAGGCAGGCGGCGAAGGCCATGACGTGCGCTATGA
AGGTCTGCTCCTGCACGCCGTGAACCAGGTGCGCCTGCGGGCCGCGCGC
GAACACCGCCACGTCCTCGCCTGCGTGGGTCTCTTCGTCCAGGGGCACT
GCTGACTGCTGCCGATACTCGGGGCTCCCGCTCTCGCTCTCGGTAACAT
CCGGCCGGGCGCCGTCCTTGAGCACATAGCCTGGACCGTTTC.

In some embodiments, polynucleotides described herein may comprise one or more regulatory elements. A person of ordinary skill in the art can select regulatory elements for use in appropriate host cells, for example, mammalian or human host cells. Regulatory elements include, for example, promoters, transcription termination sequences, translation termination sequences, enhancers, and polyadenylation elements. A polynucleotide described herein may comprise a promoter sequence operably linked to a nucleotide sequence encoding a desired polypeptide, such as Otoferlin. Promoters contemplated for use in the subject invention include, but are not limited to, cytomegalovirus (CMV) promoter, SV40 promoter, Rous sarcoma virus (RSV) promoter, chimeric CMV/chicken β actin promoter (CBA) and the truncated form of CBA (smCBA) (see, e.g., Haire et al. 2006 and U.S. Pat. No. 8,298,818, which is specifically incorporated herein in its entirety by express reference thereto). In some embodiments, the promoter is the truncated chimeric CMV ß actin (smcBA) promoter. In some embodiments, the promoter comprises a sequence that is at least 75%, at least 80%, at least 85%, at least 90%, at least 95%, at least 96%, at least 97%, at least 98%, at least 99% or 100% identical with the nucleotide sequence

(SEQ ID NO: 4)
GGTACCCTAGTTATTAATAGTAATCAATTACGGGGTCATTAGTTCATAG
CCCATATATGGAGTTCCGCGTTACATAACTTACGGTAAATGGCCCGCCT
GGCTGACCGCCCAACGACCCCCGCCCATTGACGTCAATAATGACGTATG
TTCCCATAGTAACGCCAATAGGGACTTTCCATTGACGTCAATGGGTGGA
CTATTTACGGTAAACTGCCCACTTGGCAGTACATCAAGTGTATCATATG
CCAAGTACGCCCCCTATTGACGTCAATGACGGTAAATGGCCCGCCTGGC
ATTATGCCCAGTACATGACCTTATGGGACTTTCCTACTTGGCAGTACAT
CTACGTATTAGTCATCGCTATTACCATGGTCGAGGTGAGCCCCACGTTC
TGCTTCACTCTCCCCATCTCCCCCCCCTCCCCACCCCCAATTTTGTATT
TATTTATTTTTTAATTATTTTGTGCAGCGATGGGGGCGGGGGGGGGGGG
GGGGCGCGCGCCAGGCGGGGCGGGGCGGGGCGAGGGGCGGGGCGGGGCG
AGGCGGAGAGGTGCGGCGGCAGCCAATCAGAGCGGCGCGCTCCGAAAGT
TTCCTTTTATGGCGAGGCGGCGGCGGCGGCGGCCCTATAAAAAGCGAAG
CGCGCGGCGGGCG.

In some embodiments, polynucleotides as described herein comprise a partial coding sequence that encodes an N-terminal or C-terminal portion of an Otoferlin polypeptide, wherein the partial coding sequences can be spliced or otherwise combined together in vivo in order to encode an Otoferlin polypeptide. In some embodiments, the Otoferlin polypeptide is a human Otoferlin polypeptide. In some embodiments, the Otoferlin polypeptide is a long isoform of a human Otoferlin polypeptide (see, e.g., Yasunaga et al. OTOF Encodes Multiple Long and Short Isoforms: Genetic Evidence That the Long Ones Underlie Recessive Deafness DFNB9. Am. J. Hum. Genet. 67:591-600, 2000). In some embodiments, the Otoferlin polypeptide comprises a sequence that is at least 75%, at least 80%, at least 85%, at least 90%, at least 95%, at least 96%, at least 97%, at least 98%, at least 99% or 100% identical with one or both of the following amino acid sequences:

Human OTOF isoform 1-Genbank Number AF183185.1
(SEQ ID NO: 5)
MALLIHLKTVSELRGRGDRIAKVTFRGQSFYSRVLENCEDVADFDETFR
WPVASSIDRNEMLEIQVFNYSKVFSNKLIGTFRMVLQKVVEESHVEVTD
TLIDDNNAIIKTSLCVEVRYQATDGTVGSWDDGDFLGDESLQEEEKDSQ
ETDGLLPGSRPSSRPPGEKSFRRAGRSVFSAMKLGKNRSHKEEPQRPDE
PAVLEMEDLDHLAIRLGDGLDPDSVSLASVTALTTNVSNKRSKPDIKME
PSAGRPMDYQVSITVIEARQLVGLNMDPVVCVEVGDDKKYTSMKESTNC
PYYNEYFVFDFHVSPDVMFDKIIKISVIHSKNLLRSGTLVGSFKMDVGT
VYSQPEHQFHHKWAILSDPDDISSGLKGYVKCDVAVVGKGDNIKTPHKA
NETDEDDIEGNLLLPEGVPPERQWARFYVKIYRAEGLPRMNTSLMANVK
KAFIGENKDLVDPYVQVFFAGQKGKTSVQKSSYEPLWNEQVVFTDLFPP
LCKRMKVQIRDSDKVNDVAIGTHFIDLRKISNDGDKGFLPTLGPAWVNM
YGSTRNYTLLDEHQDLNEGLGEGVSFRARLLLGLAVEIVDTSNPELTSS
TEVQVEQATPISESCAGKMEEFFLFGAFLEASMIDRRNGDKPITFEVTI
GNYGNEVDGLSRPQRPRPRKEPGDEEEVDLIQNASDDEAGDAGDLASVS
STPPMRPQVTDRNYFHLPYLERKPCIYIKSWWPDQRRRLYNANIMDHIA
DKLEEGLNDIQEMIKTEKSYPERRLRGVLEELSCGCCRFLSLADKDQGH
SSRTRLDRERLKSCMRELENMGQQARMLRAQVKRHTVRDKLRLCQNFLQ
KLRFLADEPQHSIPDIFIWMMSNNKRVAYARVPSKDLLFSIVEEETGKD
CAKVKTLFLKLPGKRGFGSAGWTVQAKVELYLWLGLSKQRKEFLCGLPC
GFQEVKAAQGLGLHAFPPVSLVYTKKQAFQLRAHMYQARSLFAADSSGL
SDPFARVFFINQSQCTEVLNETLCPTWDQMLVFDNLELYGEAHELRDDP
PIIVIEIYDQDSMGKADFMGRTFAKPLVKMADEAYCPPRFPPQLEYYQI
YRGNATAGDLLAAFELLQIGPAGKADLPPINGPVDVDRGPIMPVPMGIR
PVLSKYRVEVLFWGLRDLKRVNLAQVDRPRVDIECAGKGVQSSLIHNYK
KNPNFNTLVKWFEVDLPENELLHPPLNIRVVDCRAFGRYTLVGSHAVSS
LRRFIYRPPDRSAPSWNTTVRLLRRCRVLCNGGSSSHSTGEVVVTMEPE
VPIKKLETMVKLDATSEAVVKVDVAEEEKEKKKKKKGTAEEPEEEEPDE
SMLDWWSKYFASIDTMKEQLRQQEPSGIDLEEKEEVDNTEGLKGSMKGK
EKARAAKEEKKKKTQSSGSGQGSEAPEKKKPKIDELKVYPKELESEFDN
FEDWLHTFNLLRGKTGDDEDGSTEEERIVGRFKGSLCVYKVPLPEDVSR
EAGYDSTYGMFQGIPSNDPINVLVRVYVVRATDLHPADINGKADPYIAI
RLGKTDIRDKENYISKQLNPVFGKSFDIEASFPMESMLTVAVYDWDLVG
TDDLIGETKIDLENRFYSKHRATCGIAQTYSTHGYNIWRDPMKPSQILT
RLCKDGKVDGPHFGPPGRVKVANRVFTGPSEIEDENGQRKPTDEHVALL
ALRHWEDIPRAGCRLVPEHVETRPLLNPDKPGIEQGRLELWVDMFPMDM
PAPGTPLDISPRKPKKYELRVIIWNTDEVVLEDDDFFTGEKSSDIFVRG
WLKGQQEDKQDTDVHYHSLTGEGNFNWRYLFPFDYLAAEEKIVISKKES
MFSWDETEYKIPARLTLQIWDADHFSADDFLGAIELDLNRFPRGAKTAK
QCTMEMATGEVDVPLVSIFKQKRVKGWWPLLARNENDEFELTGKVEAEL
HLLTAEEAEKNPVGLARNEPDPLEKPNRPDTSFIWFLNPLKSARYFLWH
TYRWLLLKLLLLLLLLLLLALFLYSVPGYLVKKILGA
Human OTOF isoform 5-Genbank Number NP_001274418
(SEQ ID NO: 6)
MALLIHLKTVSELRGRGDRIAKVTFRGQSFYSRVLENCEDVADFDETFR
WPVASSIDRNEMLEIQVFNYSKVFSNKLIGTFRMVLQKVVEESHVEVTD
TLIDDNNAIIKTSLCVEVRYQATDGTVGSWDDGDFLGDESLQEEEKDSQ
ETDGLLPGSRPSSRPPGEKSFRRAGRSVFSAMKLGKNRSHKEEPQRPDE
PAVLEMEDLDHLAIRLGDGLDPDSVSLASVTALTTNVSNKRSKPDIKME
PSAGRPMDYQVSITVIEARQLVGLNMDPVVCVEVGDDKKYTSMKESTNC
PYYNEYFVFDFHVSPDVMFDKIIKISVIHSKNLLRSGTLVGSFKMDVGT
VYSQPEHQFHHKWAILSDPDDISSGLKGYVKCDVAVVGKGDNIKTPHKA
NETDEDDIEGNLLLPEGVPPERQWARFYVKIYRAEGLPRMNTSLMANVK
KAFIGENKDLVDPYVQVFFAGQKGKTSVQKSSYEPLWNEQVVFTDLFPP
LCKRMKVQIRDSDKVNDVAIGTHFIDLRKISNDGDKGFLPTLGPAWVNM
YGSTRNYTLLDEHQDLNEGLGEGVSFRARLLLGLAVEIVDTSNPELTSS
TEVQVEQATPISESCAGKMEEFFLFGAFLEASMIDRRNGDKPITFEVTI
GNYGNEVDGLSRPQRPRPRKEPGDEEEVDLIQNASDDEAGDAGDLASVS
STPPMRPQVTDRNYFHLPYLERKPCIYIKSWWPDQRRRLYNANIMDHIA
DKLEEGLNDIQEMIKTEKSYPERRLRGVLEELSCGCCRFLSLADKDQGH
SSRTRLDRERLKSCMRELENMGQQARMLRAQVKRHTVRDKLRLCQNFLQ
KLRFLADEPQHSIPDIFIWMMSNNKRVAYARVPSKDLLFSIVEEETGKD
CAKVKTLFLKLPGKRGFGSAGWTVQAKVELYLWLGLSKQRKEFLCGLPC
GFQEVKAAQGLGLHAFPPVSLVYTKKQAFQLRAHMYQARSLFAADSSGL
SDPFARVFFINQSQCTEVLNETLCPTWDQMLVFDNLELYGEAHELRDDP
PIIVIEIYDQDSMGKADFMGRTFAKPLVKMADEAYCPPRFPPQLEYYQI
YRGNATAGDLLAAFELLQIGPAGKADLPPINGPVDVDRGPIMPVPMGIR
PVLSKYRVEVLFWGLRDLKRVNLAQVDRPRVDIECAGKGVQSSLIHNYK
KNPNFNTLVKWFEVDLPENELLHPPLNIRVVDCRAFGRYTLVGSHAVSS
LRRFIYRPPDRSAPSWNTTVRLLRRCRVLCNGGSSSHSTGEVVVTMEPE
VPIKKLETMVKLDATSEAVVKVDVAEEEKEKKKKKKGTAEEPEEEEPDE
SMLDWWSKYFASIDTMKEQLRQQEPSGIDLEEKEEVDNTEGLKGSMKGK
EKARAAKEEKKKKTQSSGSGQGSEAPEKKKPKIDELKVYPKELESEFDN
FEDWLHTFNLLRGKTGDDEDGSTEEERIVGRFKGSLCVYKVPLPEDVSR
EAGYDSTYGMFQGIPSNDPINVLVRVYVVRATDLHPADINGKADPYIAI
RLGKTDIRDKENYISKQLNPVFGKSFDIEASFPMESMLTVAVYDWDLVG
TDDLIGETKIDLENRFYSKHRATCGIAQTYSTHGYNIWRDPMKPSQILT
RLCKDGKVDGPHFGPPGRVKVANRVFTGPSEIEDENGQRKPTDEHVALL
ALRHWEDIPRAGCRLVPEHVETRPLLNPDKPGIEQGRLELWVDMFPMDM
PAPGTPLDISPRKPKKYELRVIIWNTDEVVLEDDDFFTGEKSSDIFVRG
WLKGQQEDKQDTDVHYHSLTGEGNFNWRYLFPFDYLAAEEKIVISKKES
MFSWDETEYKIPARLTLQIWDADHFSADDFLGAIELDLNRFPRGAKTAK
QCTMEMATGEVDVPLVSIFKQKRVKGWWPLLARNENDEFELTGKVEAEL
HLLTAEEAEKNPVGLARNEPDPLEKPNRPDTAFVWFLNPLKSIKYLICT
RYKWLIIKIVLALLGLLMLGLFLYSLPGYMVKKLLGA

In some embodiments, the Otoferlin polypeptide is a mouse Otoferlin polypeptide. In some embodiments, the Otoferlin polypeptide comprises a sequence that is at least 75%, at least 80%, at least 85%, at least 90%, at least 95%, at least 96%, at least 97%, at least 98%, at least 99% or 100% identical with the following amino acid sequence:

Mouse OTOF Isoform 1-Genbank Number NP_001093865.1
(SEQ ID NO: 9)
MALIVHLKTVSELRGKGDRIAKVTFRGQSFYSRVLENCEGVADFDETFR
WPVASSIDRNEVLEIQIFNYSKVFSNKLIGTFCMVLQKVVEENRVEVTD
TLMDDSNAIIKTSLSMEVRYQATDGTVGPWDDGDFLGDESLQEEKDSQE
TDGLLPGSRPSTRISGEKSFRSKGREKTKGGRDGEHKAGRSVFSAMKLG
KTRSHKEEPQRQDEPAVLEMEDLDHLAIQLGDGLDPDSVSLASVTALTS
NVSNKRSKPDIKMEPSAGRPMDYQVSITVIEARQLVGLNMDPVVCVEVG
DDKKYTSMKESTNCPYYNEYFVFDFHVSPDVMFDKIIKISVIHSKNLLR
SGTLVGSFKMDVGTVYSQPEHQFHHKWAILSDPDDISAGLKGYVKCDVA
VVGKGDNIKTPHKANETDEDDIEGNLLLPEGVPPERQWARFYVKIYRAE
GLPRMNTSLMANVKKAFIGENKDLVDPYVQVFFAGQKGKTSVQKSSYEP
LWNEQVVFTDLFPPLCKRMKVQIRDSDKVNDVAIGTHFIDLRKISNDGD
KGFLPTLGPAWVNMYGSTRNYTLLDEHQDLNEGLGEGVSFRARLMLGLA
VEILDTSNPELTSSTEVQVEQATPVSESCTGRMEEFFLFGAFLEASMID
RKNGDKPITFEVTIGNYGNEVDGMSRPLRPRPRKEPGDEEEVDLIQNSS
DDEGDEAGDLASVSSTPPMRPQITDRNYFHLPYLERKPCIYIKSWWPDQ
RRRLYNANIMDHIADKLEEGLNDVQEMIKTEKSYPERRLRGVLEELSCG
CHRFLSLSDKDQGRSSRTRLDRERLKSCMRELESMGQQAKSLRAQVKRH
TVRDKLRSCQNFLQKLRFLADEPQHSIPDVFIWMMSNNKRIAYARVPSK
DLLFSIVEEELGKDCAKVKTLFLKLPGKRGFGSAGWTVQAKLELYLWLG
LSKQRKDFLCGLPCGFEEVKAAQGLGLHSFPPISLVYTKKQAFQLRAHM
YQARSLFAADSSGLSDPFARVFFINQSQCTEVLNETLCPTWDQMLVFDN
LELYGEAHELRDDPPIIVIEIYDQDSMGKADFMGRTFAKPLVKMADEAY
CPPRFPPQLEYYQIYRGSATAGDLLAAFELLQIGPSGKADLPPINGPVD
MDRGPIMPVPVGIRPVLSKYRVEVLFWGLRDLKRVNLAQVDRPRVDIEC
AGKGVQSSLIHNYKKNPNFNTLVKWFEVDLPENELLHPPLNIRVVDCRA
FGRYTLVGSHAVSSLRRFIYRPPDRSAPNWNTTGEVVVSMEPEEPVKKL
ETMVKLDATSDAVVKVDVAEDEKERKKKKKKGPSEEPEEEEPDESMLDW
WSKYFASIDTMKEQLRQHETSGTDLEEKEEMESAEGLKGPMKSKEKSRA
AKEEKKKKNQSPGPGQGSEAPEKKKAKIDELKVYPKELESEFDSFEDWL
HTFNLLRGKTGDDEDGSTEEERIVGRFKGSLCVYKVPLPEDVSREAGYD
PTYGMFQGIPSNDPINVLVRIYVVRATDLHPADINGKADPYIAIKLGKT
DIRDKENYISKQLNPVFGKSFDIEASFPMESMLTVAVYDWDLVGTDDLI
GETKIDLENRFYSKHRATCGIAQTYSIHGYNIWRDPMKPSQILTRLCKE
GKVDGPHFGPHGRVRVANRVFTGPSEIEDENGQRKPTDEHVALSALRHW
EDIPRVGCRLVPEHVETRPLLNPDKPGIEQGRLELWVDMFPMDMPAPGT
PLDISPRKPKKYELRVIVWNTDEVVLEDDDFFTGEKSSDIFVRGWLKGQ
QEDKQDTDVHYHSLTGEGNFNWRYLFPFDYLAAEEKIVMSKKESMFSWD
ETEYKIPARLTLQIWDADHFSADDFLGAIELDLNRFPRGAKTAKQCTME
MATGEVDVPLVSIFKQKRVKGWWPLLARNENDEFELTGKVEAELHLLTA
EEAEKNPVGLARNEPDPLEKPNRPDTAFVWFLNPLKSIKYLICTRYKWL
IIKIVLALLGLLMLALFLYSLPGYMVKKLLGA

In some embodiments, polynucleotides described herein comprise a splice donor or splice acceptor site. In some embodiments, the splice donor and/or splice acceptor sites contain splice consensus sequences. In some embodiments, the splice donor and/or splice acceptor sites contain sequences splice consensus sequences derived from alkaline phosphatase. In some embodiments, the splice donor site comprises a sequence that is at least 75%, at least 80%, at least 85%, at least 90%, at least 95%, at least 96%, at least 97%, at least 98%, at least 99% or 100% identical with the nucleotide sequence

GTAAGTATCAAGGTTACAAGACAGGTTTAAGGAGACCAATAGAAACTGGGCTTGTC GAGACAGAGAAGACTCTTGCGTTTCTGA (SEQ ID NO: 7). In some embodiments, the splice acceptor site comprises a sequence that is at least 75%, at least 80%, at least 85%, at least 90%, at least 95%, at least 96%, at least 97%, at least 98%, at least 99% or 100% identical with the nucleotide sequence

(SEQ ID NO: 8)
TAGGCACCTATTGGTCTTACTGACATCCACTTTGCCTTTCTCTCCACAG.

In some embodiments, polynucleotides described herein comprise ITR sequences. The ITR sequences of a polynucleotide described herein can be derived from any AAV serotype (e.g., 1, 2, 3, 4, 5, 6, 7, 8, 9, or 10) or can be derived from more than one serotype. In some embodiments of the polynucleotide provided herein, the ITR sequences are derived from AAV2. ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference). An exemplary AAV2 ITR sequence for flanking the 5′ end of an expression construct comprises the sequence:

TTGGCCACTCCCTCTCTGCGCGCTCGCTCGCTCACTGAGGCCGGGCGACCAAAGGTC GCCCGACGCCCGGGCTTTGCCCGGGCGGCCTCAGTGAGCGAGCGAGCGCGCAGAGA GGGAGTGGCCAACTCCATCACTAGGGGTTC (SEQ ID NO: 10). An exemplary AAV2 ITR sequence for flanking the 3′ end of an expression construct comprises the sequence

(SEQ ID NO: 11)
ACCCCTAGTGATGGAGTTGGCCACTCCCTCTCTGCGCGCTCGCTCGCT
CACTGAGGCCGCCCGGGCAAAGCCCGGGCGTCGGGCGACCTTTGGTCG
CCCGGCCTCAGTGAGCGAGCGAGCGCGCAGAGAGGGAGTGGCCAACC.

In some embodiments, polynucleotides described herein may further optionally include one or more transcription termination sequences, one or more translation termination sequences, one or more signal peptide sequences, one or more internal ribosome entry sites (IRES), and/or one or more enhancer elements, or any combination thereof. Transcription termination regions can typically be obtained from the 3′ untranslated region of a eukaryotic or viral gene sequence. Transcription termination sequences can be positioned downstream of a coding sequence to provide for efficient termination. Signal peptide sequences are amino-terminal peptidic sequences that encode information responsible for the location of an operably-linked polypeptide to one or more post-translational cellular destinations, including, for example, specific organelle compartments, or to the sites of protein synthesis and/or activity, and even to the extracellular environment. In some embodiments, a polynucleotide as described herein comprises a bovine growth hormone polyadenylation signal.

In some embodiments, the expression constructs contained within the polynucleotides described herein are no more than 5 kilobases, no more than 4 kilobases, or no more than 3 kilobases in size. In some embodiments, the expression construct is between 4 and 5 kilobases in size.

In some embodiments, polynucleotides described herein are contained within one or more recombinant AAV particles (e.g., first and second AAV particles). The AAV particles may be of any AAV serotype (e.g., 1, 2, 3, 4, 5, 6, 7, 8, 9, or 10), including any derivative (including non-naturally occurring variants of a serotype) or pseudotype. Non-limiting examples of derivatives and pseudotypes include AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV218, AAV-HSC15/17, AAVM41, AAV9.45, AAV6 (Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20 (4): 699-708. doi: 10.1038/mt.2011.287. Epub 2012 Jan. 24. The AAV vector toolkit: poised at the clinical crossroads. Asokan Al, Schaffer D V, Samulski R J.). In some embodiments, the first and second AAV particle are AAV2 serotype particles.

Methods of producing AAV particles and polynucleotides are known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, the polynucleotides (e.g., as plasmids) may be combined with one or more helper plasmids, e.g., that contain a rep gene (e.g., encoding Rep78, Rep68, Rep52 and Rep40) and a cap gene (encoding VP1, VP2, and VP3), and transfected into a producer cell line such that the AAV particle can be packaged and subsequently purified.

In some embodiments, the one or more helper plasmids includes a first helper plasmid comprising a rep gene and a cap gene and a second helper plasmid comprising other genes that assist in AAV production, such as a E1a gene, a E1b gene, a E4 gene, a E2a gene, and a VA gene. In some embodiments, the rep gene is a rep gene derived from AAV2. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG (R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adenoassociated viral vector reference standards, Molecular Therapy, Vol. 16, 1185-1188).

An exemplary, non-limiting, AAV particle production method is described next. One or more helper plasmids are produced or obtained, which comprise rep and cap ORFs for the desired AAV serotype and the adenoviral VA, E2A (DBP), and E4 genes under the transcriptional control of their native promoters. HEK293 cells (available from ATCC®) are transfected via CaPO₄-mediated transfection, lipids or polymeric molecules such as Polyethylenimine (PEI) with the helper plasmid(s) and a plasmid containing a polynucleotide described herein. Alternatively, in another non-limiting example, Sf9-based producer stable cell lines are infected with a single recombinant baculovirus containing the polynucleotide. As a further non-limiting alternative, in another example HEK293 or BHK cell lines are infected with a HSV containing the polynucleotide and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for AAV particle production. The AAV particles can then be purified using any method known in the art or described herein, e.g., by iodixanol step gradient, CsCl gradient, chromatography, or polyethylene glycol (PEG) precipitation.

The disclosure also contemplates host cells that comprise at least one of the disclosed AAV particles or polynucleotides. Such host cells include mammalian host cells, with human host cells being preferred, and may be either isolated, in cell or tissue culture. In the case of genetically modified animal models (e.g., a mouse), the transformed host cells may be comprised within the body of a non-human animal itself.

### Methods and Subjects

In some aspects, methods of increasing expression of Otoferlin in a cell are provided. In some embodiments, the method comprises contacting the cell with a first AAV particle as described herein comprising a first polynucleotide as described herein; and contacting the cell with a second AAV particle as described herein comprising a second polynucleotide as described herein. In some embodiments, the cell is a mammalian cell such as a mouse or human cell. In some embodiments, the cell is ex vivo. In some embodiments, the cell is in vivo. In some embodiments, the cell is a cell of the ear (e.g., the cell of a human ear). In some embodiments, the cell is a cell of the inner ear (e.g., the cell of a human inner ear). In some embodiments, the cell is in a subject (e.g., a mammalian subject such as a human subject).

Other aspects of the disclosure relate to treatment of a disease or condition caused by decreased or absent expression or activity of Otoferlin. In some embodiments, the method comprises administering to a subject a therapeutically effective amount of a first AAV particle as described herein comprising a first polynucleotide as described herein and a therapeutically effective amount of a second AAV particle as described herein comprising a second polynucleotide as described herein. In some embodiments, the subject is a human subject and the subject has Deafness, Autosomal Recessive 9 (DFNB9). In some embodiments, the subject is a human subject having impaired vestibular function or a vestibular disorder (see, e.g., Dulon et al. Otoferlin is Critical for a Highly Sensitive and Linear Calcium Dependent Exocytosis at Vestibular Hair Cell Ribbon Synapses. J Neurosci. 2009; 29(34): 10474-10487).

To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of AAV particles may be an amount of the particles that are capable of transferring an expression construct to a host organ, tissue, or cell. A therapeutically acceptable amount may be an amount that is capable of treating a disease, e.g., DFNB9. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

The AAV particles or polynucleotides may be delivered in the form of a composition, such as a composition comprising the active ingredient, such as AAV particles described herein, and a pharmaceutically acceptable carrier as described herein. The AAV particles or polynucleotides may be prepared in a variety of compositions, and may also be formulated in appropriate pharmaceutical vehicles for administration to human or animal subjects. In some embodiments, where first and second AAV particles are utilized, the first and second AAV particles may be contained within the same composition or within different compositions and may be administered together or separately.

In some embodiments, the AAV particles administered to a subject may be provided in a composition having a concentration on the order ranging from 10⁶ to 10¹⁴ particles/ml or 10³ to 10¹⁵ particles/ml, or any values there between for either range, such as for example, about 10⁶, 10⁷, 10⁸, 10⁹, 10¹⁰, 10¹¹, 10¹², 10¹³, or 10¹⁴ particles/ml. In one embodiment, AAV particles of higher than 10¹³ particles/ml are be administered. In some embodiments, the number of AAV particles administered to a subject may be on the order ranging from 10⁶ to 10¹⁴ vector genomes (vgs)/ml or 10³ to 10¹⁵ vgs/ml, or any values therebetween for either range, such as for example, about 10⁶, 10⁷, 10⁸, 10⁹, 10¹⁰, 10¹¹, 10¹², 10¹³, or 10¹⁴ vgs/ml. In one embodiment, AAV particles of higher than 10¹³ vgs/ml are be administered. The AAV particles can be administered as a single dose, or divided into two or more administrations as may be required to achieve therapy of the particular disease or disorder being treated. In some embodiments, 0.0001 ml to 10 mls are delivered to a subject. In some embodiments, the number of AAV particles administered to a subject may be on the order ranging from 10⁶-10¹⁴ vg/kg, or any values therebetween, such as for example, about 10⁶, 10⁷, 10⁸, 10⁹, 10¹⁰, 10¹¹, 10¹², 10¹³, or 10¹⁴ vgs/kg. In some embodiments, when a first AAV particle comprising a first polynucleotide as described herein and second AAV particle comprising a second polynucleotide as described herein are administered, the amount administered is the same for both particles. In some embodiments, when a first AAV particle comprising a first polynucleotide as described herein and second AAV particle comprising a second polynucleotide as described herein are administered, the amount administered is different for each particle.

If desired, AAV particles may be administered in combination with other agents or treatments as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The AAV particles may thus be delivered along with various other agents or treatments as required in the particular instance. In some embodiments, AAV particle treatment may be accompanied by use of a hearing aid.

In certain circumstances it will be desirable to deliver the AAV particles in suitably formulated pharmaceutical compositions disclosed herein either subcutaneously, parenterally, intravenously, intramuscularly, intraperitoneally, by oral or nasal inhalation, or by direct injection to one or more cells, tissues, or organs. In some embodiments, the administration is a route suitable for systemic delivery, such as by intravenous injection or infusion. In some embodiments, the administration is to the ear, e.g., via intra-cochlear administration. The pharmaceutical forms of the AAV particle compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subretinal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the AAV particles in the required amount in the appropriate solvent with several of the other ingredients enumerated above, as required, followed by filtered sterilization or another sterilization technique. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The amount of AAV particle or polynucleotide compositions and time of administration of such compositions will be within the purview of the skilled artisan having benefit of the present teachings. It is likely, however, that the administration of therapeutically effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the AAV particle compositions, either over a relatively short, or a relatively prolonged period of time, as may be determined by the medical practitioner overseeing the administration of such compositions.

The composition may include AAV particles, either alone, or in combination with one or more additional active ingredients, which may be obtained from natural or recombinant sources or chemically synthesized.

Toxicity and efficacy of the compositions utilized in methods of the disclosure can be determined by standard pharmaceutical procedures, using either cells in culture or experimental animals to determine the LD50 (the dose lethal to 50% of the population). The dose ratio between toxicity and efficacy is the therapeutic index and it can be expressed as the ratio LD50/ED50. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

Aspects of the disclosure relate to methods for use with a subject, such as human or non-human primate subjects. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease that may be treated with gene therapy. In some embodiments, the subject has or is suspected of having Deafness, Autosomal Recessive 9 (DFNB9). DFNB9 is an autosomal recessive form of deafness though to be caused by mutations in the OTOF gene that result in a decrease in expression, functionality, or both, of the Otoferlin protein. Otoferlin protein has been shown to be important for exocytosis at the auditory ribbon synapse (see, e.g., Roux et al. Otoferlin, defective in a human deafness form, is essential for exocytosis at the auditory ribbon synapse. (2006) Cell 127(2):277-89). Subjects having DFNB9 can be identified by the skilled physician, e.g., using a combination of electrophysiologic testing of auditory brain stem responses (ABRs) and genetic testing to identify mutations in the OTOF gene (see, e.g., OMIM entries 603681 and 601071). In some embodiments, the subject is a human subject that has one or more of the following nonsense or missense mutations in the OTOF gene: TYR730TER, GLN829TER, PRO1825ALA, PRO50ARG, LEU1011PRO, ILE515THR, ARG1939GLN, or GLY541SER. In some embodiments, the subject is a human subject that has an A-to-G transition at the intron 8/exon 9 junction (IVS8-2A-G) or an G-to-A transition at position +1, the first intronic nucleotide in the splice donor site of exon 5 or a G-C transversion in the donor splice site of intron 39. In some embodiments, the subject is a human subject that has a one base pair deletion (1778G) in exon 16, leading to a stop codon, and a 6141G-A change, resulting in an ARG-to-GLN substitution in exon 48.

### Compositions

Other aspects of the disclosure relate to compositions comprising AAV particles or polynucleotides described herein. In some embodiments, AAV particles described herein are added to a composition, e.g., a pharmaceutical composition.

In some embodiments, the composition comprises a pharmaceutically acceptable carrier. The term “carrier” refers to a diluent, adjuvant, excipient, or vehicle with which the AAV particles are administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of AAV particles to human subjects. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22^{nd} edition, Pharmaceutical Press, 2012.

Typically, such compositions may contain at least about 0.1% of the therapeutic agent (e.g., AAV particles) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., AAV particles) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, a composition described herein may be administered to a subject in need thereof, such as a subject having DFNB9. In some embodiments, a method described herein may comprise administering a composition or multiple compositions comprising AAV particles as described herein to a subject in need thereof. In some embodiments, the subject is a human subject. In some embodiments, the subject has or is suspected of having a disease that may be treated with gene therapy, such as DFNB9. In some embodiments, the subject has been diagnosed with DFNB9.

### Kits

Other aspects of the disclosure relate to kits comprising AAV particles or polynucleotides as described herein in one or more containers. Kits can optionally include pharmaceutically acceptable carriers and/or diluents. In some embodiments, the kit includes instructions or packaging materials that describe how to administer AAV particles or polynucleotides contained within the kit to a selected cell or recipient. Containers of the kit can be of any suitable material, e.g., glass, plastic, metal, etc., and of any suitable size, shape, or configuration. In some embodiments, the kits may include one or more ampoules or syringes that contain AAV particles or polynucleotides in a suitable liquid or solution form.

### EXAMPLES

### Rescue of Hearing in OTOF Knock-Out Mice Using Adeno-Associated Virus Gene Therapy Approach

### Introduction

Otoferlin is the key calcium sensor for neurotransmitter release in the ear (see, e.g., Roux 2006). Otoferlin is mainly expressed in the inner hair cells of the cochlea and only few other cells of the central nervous system (see, e.g., Yasunaga et al. 1999 & 2000). It is a member of the ferlin family of transmembrane proteins which share a common C2 domain also found in synaptotagmin, PKC and PLC.

Mutations in the human OTOF gene, which encodes human Otoferlin, cause a type of nonsyndromic deafness called Deafness, Autosomal Recessive 9 (DFNB9). OTOF knock-out mice have also been shown to have severe hearing loss despite normal inner hair cell development and auditory ribbon synapse formation (see, e.g., Roux et al. (2006) Otoferlin, defective in a human deafness form, is essential for exocytosis at the auditory ribbon synapse. Cell. 127:277-289). However, Otof^{-/-} mice lose the auditory brain stem response across all sound frequencies due to complete abolishment of synaptic exocytosis and, as a consequence, abolishment of neurotransmitter release from synaptic vesicles.

DFNB9 manifests in humans as two phenotypes, as a nonsyndromic bilateral loss of hearing before the acquiring of language and less frequently as a temperature-sensitive nonsyndromic auditory neuropathy. It was first discovered in an affected Lebanese family (Chaib et al. 1996) and has since been found in many parts of the world (see, e.g., Adato et al. 2000, Rodriguez-Ballesteros et al. 2003, Choi et al. 2009, Matsunaga et al. 2012).

Current treatment in humans with DFNB9 utilizes cochlear implants and hearing aids. In addition, for the temperature-sensitive form of DFNB9, prevention of fevers and other conditions that would cause the body temperature to rise are important. Applicants sought to use adeno-associated virus (AAV) as a means to restore expression of OTOF in the knock-out mice as a proof-of-concept for using AAV to delivery OTOF as a treatment for DFNB9. The mouse OTOF cDNA is 5979 base pairs in length whereas most AAVs cannot package more than approximately 4.8 kilobases of genome. As a result, a dual vector system was used to separately deliver the 5′ portion of the cDNA and the 3′ portion of the cDNA as separate AAV constructs such that the full-length cDNA could be reassembled in vivo once delivered.

### Methods

### Dual AAV Vector Constructs

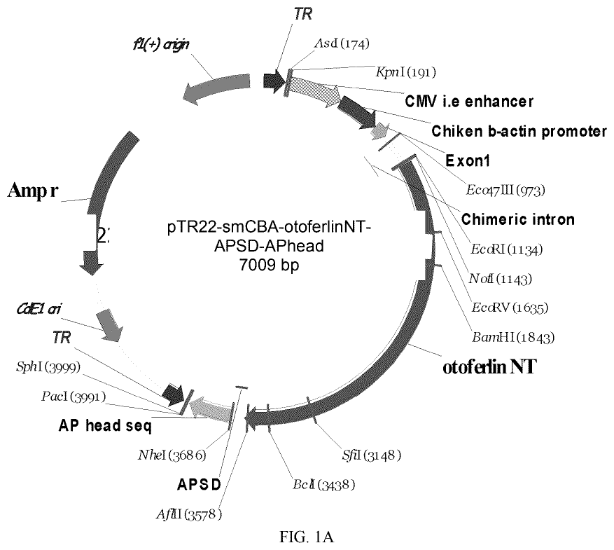

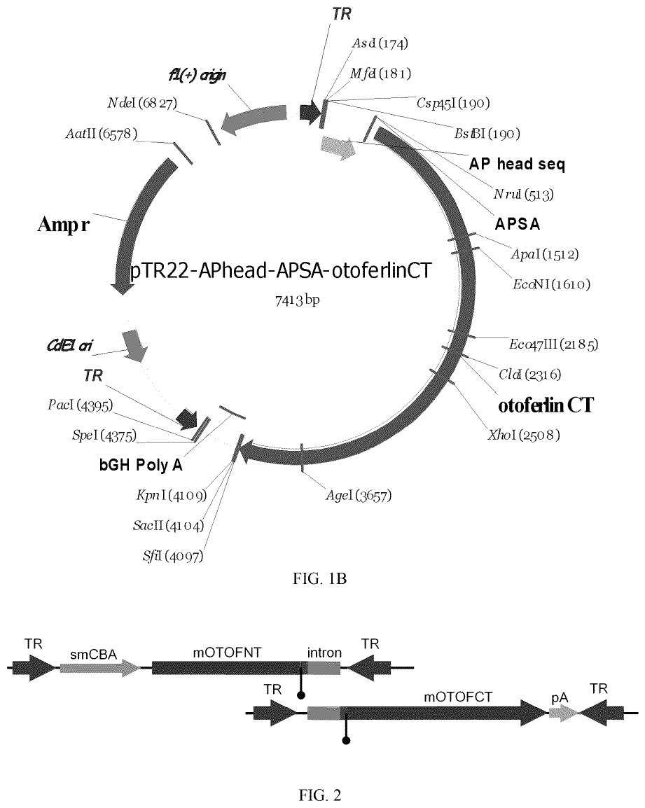

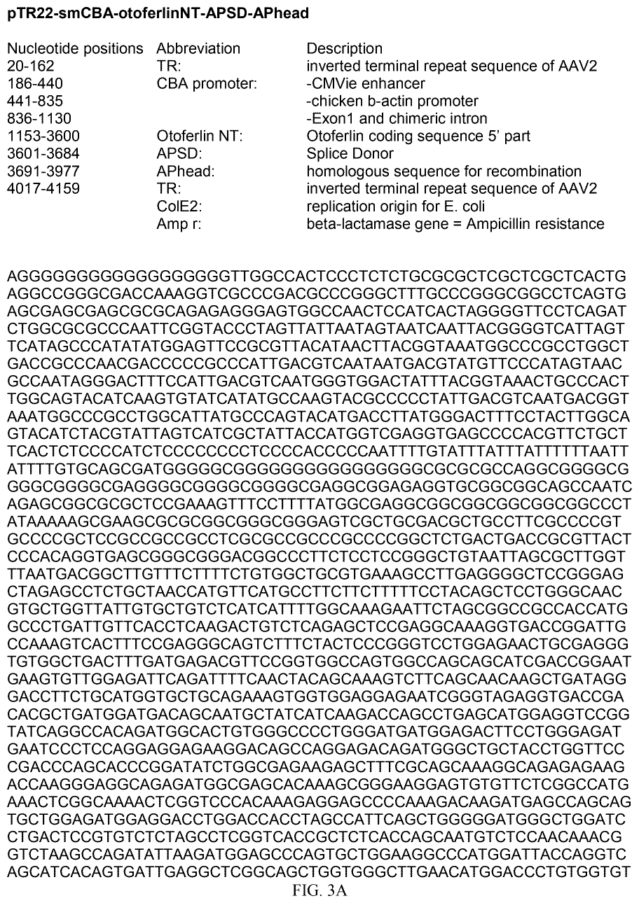

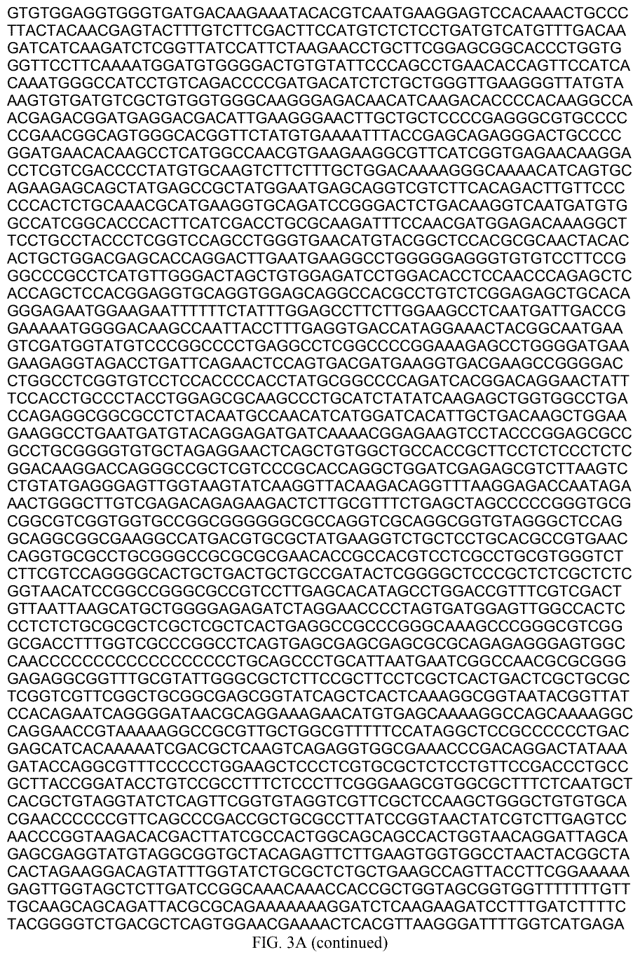

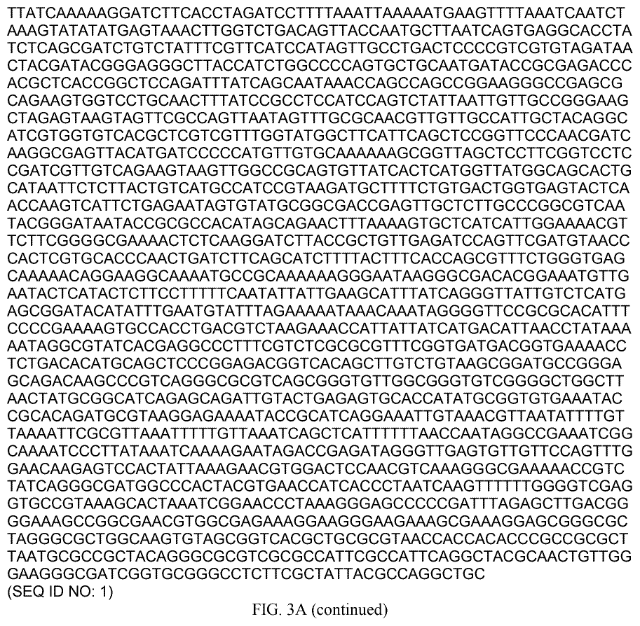

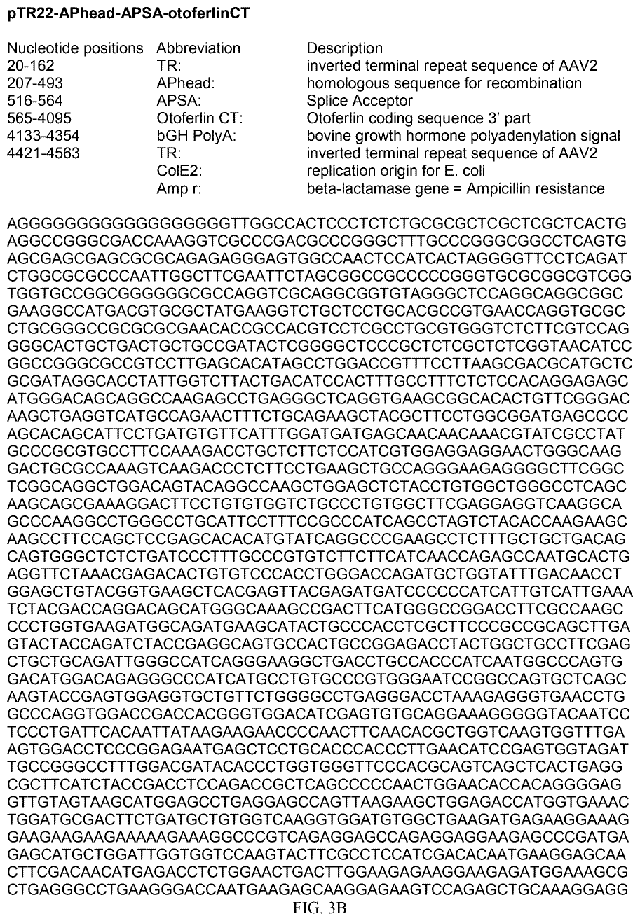

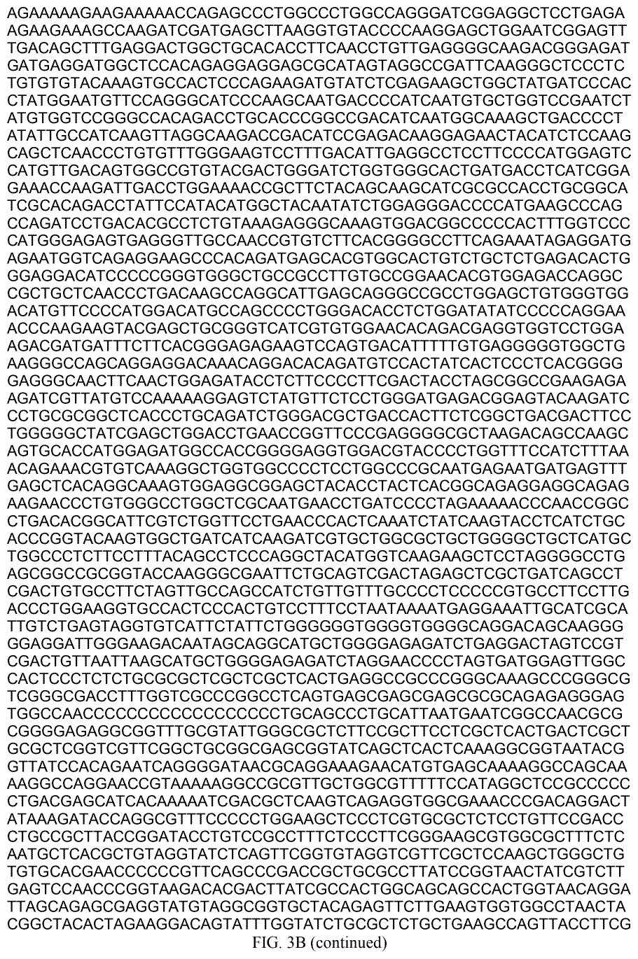

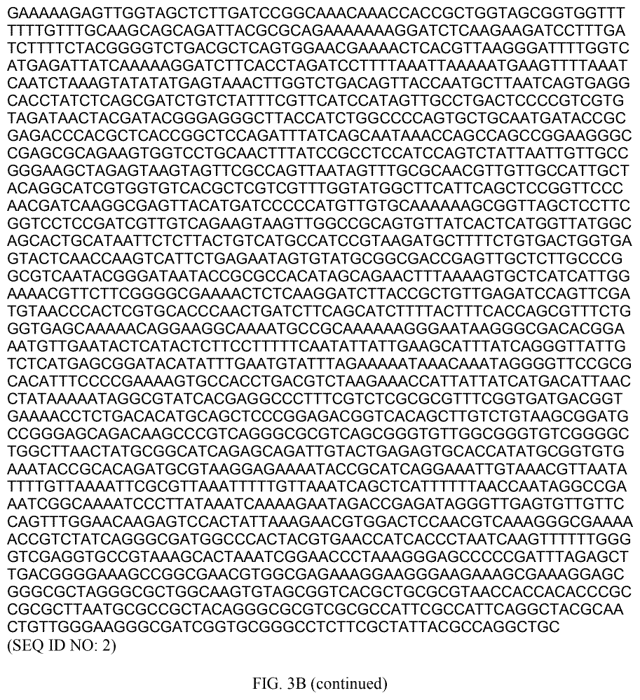

A mouse OTOF cDNA was split into two sections, a 5′ and 3′ section and inserted into two AAV ITR-containing plasmids. The sequence of each of the two cassettes in the plasmids is shown below and the maps of each construct are shown in FIGS. 1 and 2. Annotated versions of the cassettes are shown in FIGS. 3A and 3B. Each cassette contains a region of homology to promote homologous recombination between the 5′ and 3′ ends of the cDNA in vivo (see Ghosh et al., 2011). Once recombined in vivo, the full-length cDNA contains a splice donor/splice acceptor pair that causes splicing out of the region of homology. The vectors were packaged into AAV2 serotype particles using standard plasmid transfection methods as previously described (see Zolotukhin et al. Production and purification of serotype 1, 2, and 5 recombinant adeno-associated viral vectors. Methods 28 (2002) 158-167). The viral particles were purified by standard methods as previously described (see Zolotukhin et al. Production and purification of serotype 1, 2, and 5 recombinant adeno-associated viral vectors. Methods 28 (2002) 158-167). The viral particles carrying the 5′ portion of the OTOF cDNA are also referred to herein as “AAV2-OTOF-NT.” The viral particles carrying the 3′ portion of the OTOF cDNA are also referred to herein as “AAV2-OTOF-CT.”

pTR22-smCBA-otoferlinNT-APSD-APhead
(SEQ ID NO: 1)
AGGGGGGGGGGGGGGGGGGTTGGCCACTCCCTCTCTGCGCGCTCGCTC
GCTCACTGAGGCCGGGCGACCAAAGGTCGCCCGACGCCCGGGCTTTGC
CCGGGCGGCCTCAGTGAGCGAGCGAGCGCGCAGAGAGGGAGTGGCCAA
CTCCATCACTAGGGGTTCCTCAGATCTGGCGCGCCCAATTCGGTACCC
TAGTTATTAATAGTAATCAATTACGGGGTCATTAGTTCATAGCCCATA
TATGGAGTTCCGCGTTACATAACTTACGGTAAATGGCCCGCCTGGCTG
ACCGCCCAACGACCCCCGCCCATTGACGTCAATAATGACGTATGTTCC
CATAGTAACGCCAATAGGGACTTTCCATTGACGTCAATGGGTGGACTA
TTTACGGTAAACTGCCCACTTGGCAGTACATCAAGTGTATCATATGCC
AAGTACGCCCCCTATTGACGTCAATGACGGTAAATGGCCCGCCTGGCA
TTATGCCCAGTACATGACCTTATGGGACTTTCCTACTTGGCAGTACAT
CTACGTATTAGTCATCGCTATTACCATGGTCGAGGTGAGCCCCACGTT
CTGCTTCACTCTCCCCATCTCCCCCCCCTCCCCACCCCCAATTTTGTA
TTTATTTATTTTTTAATTATTTTGTGCAGCGATGGGGGCGGGGGGGGG
GGGGGGGCGCGCGCCAGGCGGGGCGGGGCGGGGCGAGGGGCGGGGCGG
GGCGAGGCGGAGAGGTGCGGCGGCAGCCAATCAGAGCGGCGCGCTCCG
AAAGTTTCCTTTTATGGCGAGGCGGCGGCGGCGGCGGCCCTATAAAAA
GCGAAGCGCGCGGCGGGCGGGAGTCGCTGCGACGCTGCCTTCGCCCCG
TGCCCCGCTCCGCCGCCGCCTCGCGCCGCCCGCCCCGGCTCTGACTGA
CCGCGTTACTCCCACAGGTGAGCGGGCGGGACGGCCCTTCTCCTCCGG
GCTGTAATTAGCGCTTGGTTTAATGACGGCTTGTTTCTTTTCTGTGGC
TGCGTGAAAGCCTTGAGGGGCTCCGGGAGCTAGAGCCTCTGCTAACCA
TGTTCATGCCTTCTTCTTTTTCCTACAGCTCCTGGGCAACGTGCTGGT
TATTGTGCTGTCTCATCATTTTGGCAAAGAATTCTAGCGGCCGCCACC
ATGGCCCTGATTGTTCACCTCAAGACTGTCTCAGAGCTCCGAGGCAAA
GGTGACCGGATTGCCAAAGTCACTTTCCGAGGGCAGTCTTTCTACTCC
CGGGTCCTGGAGAACTGCGAGGGTGTGGCTGACTTTGATGAGACGTTC
CGGTGGCCAGTGGCCAGCAGCATCGACCGGAATGAAGTGTTGGAGATT
CAGATTTTCAACTACAGCAAAGTCTTCAGCAACAAGCTGATAGGGACC
TTCTGCATGGTGCTGCAGAAAGTGGTGGAGGAGAATCGGGTAGAGGTG
ACCGACACGCTGATGGATGACAGCAATGCTATCATCAAGACCAGCCTG
AGCATGGAGGTCCGGTATCAGGCCACAGATGGCACTGTGGGCCCCTGG
GATGATGGAGACTTCCTGGGAGATGAATCCCTCCAGGAGGAGAAGGAC
AGCCAGGAGACAGATGGGCTGCTACCTGGTTCCCGACCCAGCACCCGG
ATATCTGGCGAGAAGAGCTTTCGCAGCAAAGGCAGAGAGAAGACCAAG
GGAGGCAGAGATGGCGAGCACAAAGCGGGAAGGAGTGTGTTCTCGGCC
ATGAAACTCGGCAAAACTCGGTCCCACAAAGAGGAGCCCCAAAGACAA
GATGAGCCAGCAGTGCTGGAGATGGAGGACCTGGACCACCTAGCCATT
CAGCTGGGGGATGGGCTGGATCCTGACTCCGTGTCTCTAGCCTCGGTC
ACCGCTCTCACCAGCAATGTCTCCAACAAACGGTCTAAGCCAGATATT
AAGATGGAGCCCAGTGCTGGAAGGCCCATGGATTACCAGGTCAGCATC
ACAGTGATTGAGGCTCGGCAGCTGGTGGGCTTGAACATGGACCCTGTG
GTGTGTGTGGAGGTGGGTGATGACAAGAAATACACGTCAATGAAGGAG
TCCACAAACTGCCCTTACTACAACGAGTACTTTGTCTTCGACTTCCAT
GTCTCTCCTGATGTCATGTTTGACAAGATCATCAAGATCTCGGTTATC
CATTCTAAGAACCTGCTTCGGAGCGGCACCCTGGTGGGTTCCTTCAAA
ATGGATGTGGGGACTGTGTATTCCCAGCCTGAACACCAGTTCCATCAC
AAATGGGCCATCCTGTCAGACCCCGATGACATCTCTGCTGGGTTGAAG
GGTTATGTAAAGTGTGATGTCGCTGTGGTGGGCAAGGGAGACAACATC
AAGACACCCCACAAGGCCAACGAGACGGATGAGGACGACATTGAAGGG
AACTTGCTGCTCCCCGAGGGCGTGCCCCCCGAACGGCAGTGGGCACGG
TTCTATGTGAAAATTTACCGAGCAGAGGGACTGCCCCGGATGAACACA
AGCCTCATGGCCAACGTGAAGAAGGCGTTCATCGGTGAGAACAAGGAC
CTCGTCGACCCCTATGTGCAAGTCTTCTTTGCTGGACAAAAGGGCAAA
ACATCAGTGCAGAAGAGCAGCTATGAGCCGCTATGGAATGAGCAGGTC
GTCTTCACAGACTTGTTCCCCCCACTCTGCAAACGCATGAAGGTGCAG
ATCCGGGACTCTGACAAGGTCAATGATGTGGCCATCGGCACCCACTTC
ATCGACCTGCGCAAGATTTCCAACGATGGAGACAAAGGCTTCCTGCCT
ACCCTCGGTCCAGCCTGGGTGAACATGTACGGCTCCACGCGCAACTAC
ACACTGCTGGACGAGCACCAGGACTTGAATGAAGGCCTGGGGGAGGGT
GTGTCCTTCCGGGCCCGCCTCATGTTGGGACTAGCTGTGGAGATCCTG
GACACCTCCAACCCAGAGCTCACCAGCTCCACGGAGGTGCAGGTGGAG
CAGGCCACGCCTGTCTCGGAGAGCTGCACAGGGAGAATGGAAGAATTT
TTTCTATTTGGAGCCTTCTTGGAAGCCTCAATGATTGACCGGAAAAAT
GGGGACAAGCCAATTACCTTTGAGGTGACCATAGGAAACTACGGCAAT
GAAGTCGATGGTATGTCCCGGCCCCTGAGGCCTCGGCCCCGGAAAGAG
CCTGGGGATGAAGAAGAGGTAGACCTGATTCAGAACTCCAGTGACGAT
GAAGGTGACGAAGCCGGGGACCTGGCCTCGGTGTCCTCCACCCCACCT
ATGCGGCCCCAGATCACGGACAGGAACTATTTCCACCTGCCCTACCTG
GAGCGCAAGCCCTGCATCTATATCAAGAGCTGGTGGCCTGACCAGAGG
CGGCGCCTCTACAATGCCAACATCATGGATCACATTGCTGACAAGCTG
GAAGAAGGCCTGAATGATGTACAGGAGATGATCAAAACGGAGAAGTCC
TACCCGGAGCGCCGCCTGCGGGGTGTGCTAGAGGAACTCAGCTGTGGC
TGCCACCGCTTCCTCTCCCTCTCGGACAAGGACCAGGGCCGCTCGTCC
CGCACCAGGCTGGATCGAGAGCGTCTTAAGTCCTGTATGAGGGAGTTG
GTAAGTATCAAGGTTACAAGACAGGTTTAAGGAGACCAATAGAAACTG
GGCTTGTCGAGACAGAGAAGACTCTTGCGTTTCTGAGCTAGCCCCCGG
GTGCGCGGCGTCGGTGGTGCCGGCGGGGGGCGCCAGGTCGCAGGCGGT
GTAGGGCTCCAGGCAGGCGGCGAAGGCCATGACGTGCGCTATGAAGGT
CTGCTCCTGCACGCCGTGAACCAGGTGCGCCTGCGGGCCGCGCGCGAA
CACCGCCACGTCCTCGCCTGCGTGGGTCTCTTCGTCCAGGGGCACTGC
TGACTGCTGCCGATACTCGGGGCTCCCGCTCTCGCTCTCGGTAACATC
CGGCCGGGCGCCGTCCTTGAGCACATAGCCTGGACCGTTTCGTCGACT
GTTAATTAAGCATGCTGGGGAGAGATCTAGGAACCCCTAGTGATGGAG
TTGGCCACTCCCTCTCTGCGCGCTCGCTCGCTCACTGAGGCCGCCCGG
GCAAAGCCCGGGCGTCGGGCGACCTTTGGTCGCCCGGCCTCAGTGAGC
GAGCGAGCGCGCAGAGAGGGAGTGGCCAACCCCCCCCCCCCCCCCCCT
GCAGCCCTGCATTAATGAATCGGCCAACGCGCGGGGAGAGGCGGTTTG
CGTATTGGGCGCTCTTCCGCTTCCTCGCTCACTGACTCGCTGCGCTCG
GTCGTTCGGCTGCGGCGAGCGGTATCAGCTCACTCAAAGGCGGTAATA
CGGTTATCCACAGAATCAGGGGATAACGCAGGAAAGAACATGTGAGCA
AAAGGCCAGCAAAAGGCCAGGAACCGTAAAAAGGCCGCGTTGCTGGCG
TTTTTCCATAGGCTCCGCCCCCCTGACGAGCATCACAAAAATCGACGC
TCAAGTCAGAGGTGGCGAAACCCGACAGGACTATAAAGATACCAGGCG
TTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTT
TCTCAATGCTCACGCTGTAGGTATCTCAGTTCGGTGTAGGTCGTTCGC
TCCAAGCTGGGCTGTGTGCACGAACCCCCCGTTCAGCCCGACCGCTGC
GCCTTATCCGGTAACTATCGTCTTGAGTCCAACCCGGTAAGACACGAC
TTATCGCCACTGGCAGCAGCCACTGGTAACAGGATTAGCAGAGCGAGG
TATGTAGGCGGTGCTACAGAGTTCTTGAAGTGGTGGCCTAACTACGGC
TACACTAGAAGGACAGTATTTGGTATCTGCGCTCTGCTGAAGCCAGTT
ACCTTCGGAAAAAGAGTTGGTAGCTCTTGATCCGGCAAACAAACCACC
GCTGGTAGCGGTGGTTTTTTTGTTTGCAAGCAGCAGATTACGCGCAGA
AAAAAAGGATCTCAAGAAGATCCTTTGATCTTTTCTACGGGGTCTGAC
GCTCAGTGGAACGAAAACTCACGTTAAGGGATTTTGGTCATGAGATTA
TCAAAAAGGATCTTCACCTAGATCCTTTTAAATTAAAAATGAAGTTTT
AAATCAATCTAAAGTATATATGAGTAAACTTGGTCTGACAGTTACCAA
TGCTTAATCAGTGAGGCACCTATCTCAGCGATCTGTCTATTTCGTTCA
TCCATAGTTGCCTGACTCCCCGTCGTGTAGATAACTACGATACGGGAG
GGCTTACCATCTGGCCCCAGTGCTGCAATGATACCGCGAGACCCACGC
TCACCGGCTCCAGATTTATCAGCAATAAACCAGCCAGCCGGAAGGGCC
GAGCGCAGAAGTGGTCCTGCAACTTTATCCGCCTCCATCCAGTCTATT
AATTGTTGCCGGGAAGCTAGAGTAAGTAGTTCGCCAGTTAATAGTTTG
CGCAACGTTGTTGCCATTGCTACAGGCATCGTGGTGTCACGCTCGTCG
TTTGGTATGGCTTCATTCAGCTCCGGTTCCCAACGATCAAGGCGAGTT
ACATGATCCCCCATGTTGTGCAAAAAAGCGGTTAGCTCCTTCGGTCCT
CCGATCGTTGTCAGAAGTAAGTTGGCCGCAGTGTTATCACTCATGGTT
ATGGCAGCACTGCATAATTCTCTTACTGTCATGCCATCCGTAAGATGC
TTTTCTGTGACTGGTGAGTACTCAACCAAGTCATTCTGAGAATAGTGT
ATGCGGCGACCGAGTTGCTCTTGCCCGGCGTCAATACGGGATAATACC
GCGCCACATAGCAGAACTTTAAAAGTGCTCATCATTGGAAAACGTTCT
TCGGGGCGAAAACTCTCAAGGATCTTACCGCTGTTGAGATCCAGTTCG
ATGTAACCCACTCGTGCACCCAACTGATCTTCAGCATCTTTTACTTTC
ACCAGCGTTTCTGGGTGAGCAAAAACAGGAAGGCAAAATGCCGCAAAA
AAGGGAATAAGGGCGACACGGAAATGTTGAATACTCATACTCTTCCTT
TTTCAATATTATTGAAGCATTTATCAGGGTTATTGTCTCATGAGCGGA
TACATATTTGAATGTATTTAGAAAAATAAACAAATAGGGGTTCCGCGC
ACATTTCCCCGAAAAGTGCCACCTGACGTCTAAGAAACCATTATTATC
ATGACATTAACCTATAAAAATAGGCGTATCACGAGGCCCTTTCGTCTC
GCGCGTTTCGGTGATGACGGTGAAAACCTCTGACACATGCAGCTCCCG
GAGACGGTCACAGCTTGTCTGTAAGCGGATGCCGGGAGCAGACAAGCC
CGTCAGGGCGCGTCAGCGGGTGTTGGCGGGTGTCGGGGCTGGCTTAAC
TATGCGGCATCAGAGCAGATTGTACTGAGAGTGCACCATATGCGGTGT
GAAATACCGCACAGATGCGTAAGGAGAAAATACCGCATCAGGAAATTG
TAAACGTTAATATTTTGTTAAAATTCGCGTTAAATTTTTGTTAAATCA
GCTCATTTTTTAACCAATAGGCCGAAATCGGCAAAATCCCTTATAAAT
CAAAAGAATAGACCGAGATAGGGTTGAGTGTTGTTCCAGTTTGGAACA
AGAGTCCACTATTAAAGAACGTGGACTCCAACGTCAAAGGGCGAAAAA
CCGTCTATCAGGGCGATGGCCCACTACGTGAACCATCACCCTAATCAA
GTTTTTTGGGGTCGAGGTGCCGTAAAGCACTAAATCGGAACCCTAAAG
GGAGCCCCCGATTTAGAGCTTGACGGGGAAAGCCGGCGAACGTGGCGA
GAAAGGAAGGGAAGAAAGCGAAAGGAGCGGGCGCTAGGGCGCTGGCAA
GTGTAGCGGTCACGCTGCGCGTAACCACCACACCCGCCGCGCTTAATG
CGCCGCTACAGGGCGCGTCGCGCCATTCGCCATTCAGGCTACGCAACT
GTTGGGAAGGGCGATCGGTGCGGGCCTCTTCGCTATTACGCCAGGCTG
C
pTR22-APhead-APSA-otoferlinCT
(SEQ ID NO: 2)
AGGGGGGGGGGGGGGGGGGTTGGCCACTCCCTCTCTGCGCGCTCGCTC
GCTCACTGAGGCCGGGCGACCAAAGGTCGCCCGACGCCCGGGCTTTGC
CCGGGCGGCCTCAGTGAGCGAGCGAGCGCGCAGAGAGGGAGTGGCCAA
CTCCATCACTAGGGGTTCCTCAGATCTGGCGCGCCCAATTGGCTTCGA
ATTCTAGCGGCCGCCCCCGGGTGCGCGGCGTCGGTGGTGCCGGCGGGG
GGCGCCAGGTCGCAGGCGGTGTAGGGCTCCAGGCAGGCGGCGAAGGCC
ATGACGTGCGCTATGAAGGTCTGCTCCTGCACGCCGTGAACCAGGTGC
GCCTGCGGGCCGCGCGCGAACACCGCCACGTCCTCGCCTGCGTGGGTC
TCTTCGTCCAGGGGCACTGCTGACTGCTGCCGATACTCGGGGCTCCCG
CTCTCGCTCTCGGTAACATCCGGCCGGGCGCCGTCCTTGAGCACATAG
CCTGGACCGTTTCCTTAAGCGACGCATGCTCGCGATAGGCACCTATTG
GTCTTACTGACATCCACTTTGCCTTTCTCTCCACAGGAGAGCATGGGA
CAGCAGGCCAAGAGCCTGAGGGCTCAGGTGAAGCGGCACACTGTTCGG
GACAAGCTGAGGTCATGCCAGAACTTTCTGCAGAAGCTACGCTTCCTG
GCGGATGAGCCCCAGCACAGCATTCCTGATGTGTTCATTTGGATGATG
AGCAACAACAAACGTATCGCCTATGCCCGCGTGCCTTCCAAAGACCTG
CTCTTCTCCATCGTGGAGGAGGAACTGGGCAAGGACTGCGCCAAAGTC
AAGACCCTCTTCCTGAAGCTGCCAGGGAAGAGGGGCTTCGGCTCGGCA
GGCTGGACAGTACAGGCCAAGCTGGAGCTCTACCTGTGGCTGGGCCTC
AGCAAGCAGCGAAAGGACTTCCTGTGTGGTCTGCCCTGTGGCTTCGAG
GAGGTCAAGGCAGCCCAAGGCCTGGGCCTGCATTCCTTTCCGCCCATC
AGCCTAGTCTACACCAAGAAGCAAGCCTTCCAGCTCCGAGCACACATG
TATCAGGCCCGAAGCCTCTTTGCTGCTGACAGCAGTGGGCTCTCTGAT
CCCTTTGCCCGTGTCTTCTTCATCAACCAGAGCCAATGCACTGAGGTT
CTAAACGAGACACTGTGTCCCACCTGGGACCAGATGCTGGTATTTGAC
AACCTGGAGCTGTACGGTGAAGCTCACGAGTTACGAGATGATCCCCCC
ATCATTGTCATTGAAATCTACGACCAGGACAGCATGGGCAAAGCCGAC
TTCATGGGCCGGACCTTCGCCAAGCCCCTGGTGAAGATGGCAGATGAA
GCATACTGCCCACCTCGCTTCCCGCCGCAGCTTGAGTACTACCAGATC
TACCGAGGCAGTGCCACTGCCGGAGACCTACTGGCTGCCTTCGAGCTG
CTGCAGATTGGGCCATCAGGGAAGGCTGACCTGCCACCCATCAATGGC
CCAGTGGACATGGACAGAGGGCCCATCATGCCTGTGCCCGTGGGAATC
CGGCCAGTGCTCAGCAAGTACCGAGTGGAGGTGCTGTTCTGGGGCCTG
AGGGACCTAAAGAGGGTGAACCTGGCCCAGGTGGACCGACCACGGGTG
GACATCGAGTGTGCAGGAAAGGGGGTACAATCCTCCCTGATTCACAAT
TATAAGAAGAACCCCAACTTCAACACGCTGGTCAAGTGGTTTGAAGTG
GACCTCCCGGAGAATGAGCTCCTGCACCCACCCTTGAACATCCGAGTG
GTAGATTGCCGGGCCTTTGGACGATACACCCTGGTGGGTTCCCACGCA
GTCAGCTCACTGAGGCGCTTCATCTACCGACCTCCAGACCGCTCAGCC
CCCAACTGGAACACCACAGGGGAGGTTGTAGTAAGCATGGAGCCTGAG
GAGCCAGTTAAGAAGCTGGAGACCATGGTGAAACTGGATGCGACTTCT
GATGCTGTGGTCAAGGTGGATGTGGCTGAAGATGAGAAGGAAAGGAAG
AAGAAGAAAAAGAAAGGCCCGTCAGAGGAGCCAGAGGAGGAAGAGCCC
GATGAGAGCATGCTGGATTGGTGGTCCAAGTACTTCGCCTCCATCGAC
ACAATGAAGGAGCAACTTCGACAACATGAGACCTCTGGAACTGACTTG
GAAGAGAAGGAAGAGATGGAAAGCGCTGAGGGCCTGAAGGGACCAATG
AAGAGCAAGGAGAAGTCCAGAGCTGCAAAGGAGGAGAAAAAGAAGAAA
AACCAGAGCCCTGGCCCTGGCCAGGGATCGGAGGCTCCTGAGAAGAAG
AAAGCCAAGATCGATGAGCTTAAGGTGTACCCCAAGGAGCTGGAATCG
GAGTTTGACAGCTTTGAGGACTGGCTGCACACCTTCAACCTGTTGAGG
GGCAAGACGGGAGATGATGAGGATGGCTCCACAGAGGAGGAGCGCATA
GTAGGCCGATTCAAGGGCTCCCTCTGTGTGTACAAAGTGCCACTCCCA
GAAGATGTATCTCGAGAAGCTGGCTATGATCCCACCTATGGAATGTTC
CAGGGCATCCCAAGCAATGACCCCATCAATGTGCTGGTCCGAATCTAT
GTGGTCCGGGCCACAGACCTGCACCCGGCCGACATCAATGGCAAAGCT
GACCCCTATATTGCCATCAAGTTAGGCAAGACCGACATCCGAGACAAG
GAGAACTACATCTCCAAGCAGCTCAACCCTGTGTTTGGGAAGTCCTTT
GACATTGAGGCCTCCTTCCCCATGGAGTCCATGTTGACAGTGGCCGTG
TACGACTGGGATCTGGTGGGCACTGATGACCTCATCGGAGAAACCAAG
ATTGACCTGGAAAACCGCTTCTACAGCAAGCATCGCGCCACCTGCGGC
ATCGCACAGACCTATTCCATACATGGCTACAATATCTGGAGGGACCCC
ATGAAGCCCAGCCAGATCCTGACACGCCTCTGTAAAGAGGGCAAAGTG
GACGGCCCCCACTTTGGTCCCCATGGGAGAGTGAGGGTTGCCAACCGT
GTCTTCACGGGGCCTTCAGAAATAGAGGATGAGAATGGTCAGAGGAAG
CCCACAGATGAGCACGTGGCACTGTCTGCTCTGAGACACTGGGAGGAC
ATCCCCCGGGTGGGCTGCCGCCTTGTGCCGGAACACGTGGAGACCAGG
CCGCTGCTCAACCCTGACAAGCCAGGCATTGAGCAGGGCCGCCTGGAG
CTGTGGGTGGACATGTTCCCCATGGACATGCCAGCCCCTGGGACACCT
CTGGATATATCCCCCAGGAAACCCAAGAAGTACGAGCTGCGGGTCATC
GTGTGGAACACAGACGAGGTGGTCCTGGAAGACGATGATTTCTTCACG
GGAGAGAAGTCCAGTGACATTTTTGTGAGGGGGTGGCTGAAGGGCCAG
CAGGAGGACAAACAGGACACAGATGTCCACTATCACTCCCTCACGGGG
GAGGGCAACTTCAACTGGAGATACCTCTTCCCCTTCGACTACCTAGCG
GCCGAAGAGAAGATCGTTATGTCCAAAAAGGAGTCTATGTTCTCCTGG
GATGAGACGGAGTACAAGATCCCTGCGCGGCTCACCCTGCAGATCTGG
GACGCTGACCACTTCTCGGCTGACGACTTCCTGGGGGCTATCGAGCTG
GACCTGAACCGGTTCCCGAGGGGCGCTAAGACAGCCAAGCAGTGCACC
ATGGAGATGGCCACCGGGGAGGTGGACGTACCCCTGGTTTCCATCTTT
AAACAGAAACGTGTCAAAGGCTGGTGGCCCCTCCTGGCCCGCAATGAG
AATGATGAGTTTGAGCTCACAGGCAAAGTGGAGGCGGAGCTACACCTA
CTCACGGCAGAGGAGGCAGAGAAGAACCCTGTGGGCCTGGCTCGCAAT
GAACCTGATCCCCTAGAAAAACCCAACCGGCCTGACACGGCATTCGTC
TGGTTCCTGAACCCACTCAAATCTATCAAGTACCTCATCTGCACCCGG
TACAAGTGGCTGATCATCAAGATCGTGCTGGCGCTGCTGGGGCTGCTC
ATGCTGGCCCTCTTCCTTTACAGCCTCCCAGGCTACATGGTCAAGAAG
CTCCTAGGGGCCTGAGCGGCCGCGGTACCAAGGGCGAATTCTGCAGTC
GACTAGAGCTCGCTGATCAGCCTCGACTGTGCCTTCTAGTTGCCAGCC
ATCTGTTGTTTGCCCCTCCCCCGTGCCTTCCTTGACCCTGGAAGGTGC
CACTCCCACTGTCCTTTCCTAATAAAATGAGGAAATTGCATCGCATTG
TCTGAGTAGGTGTCATTCTATTCTGGGGGGTGGGGTGGGGCAGGACAG
CAAGGGGGAGGATTGGGAAGACAATAGCAGGCATGCTGGGGAGAGATC
TGAGGACTAGTCCGTCGACTGTTAATTAAGCATGCTGGGGAGAGATCT
AGGAACCCCTAGTGATGGAGTTGGCCACTCCCTCTCTGCGCGCTCGCT
CGCTCACTGAGGCCGCCCGGGCAAAGCCCGGGCGTCGGGCGACCTTTG
GTCGCCCGGCCTCAGTGAGCGAGCGAGCGCGCAGAGAGGGAGTGGCCA
ACCCCCCCCCCCCCCCCCCTGCAGCCCTGCATTAATGAATCGGCCAAC
GCGCGGGGAGAGGCGGTTTGCGTATTGGGCGCTCTTCCGCTTCCTCGC
TCACTGACTCGCTGCGCTCGGTCGTTCGGCTGCGGCGAGCGGTATCAG
CTCACTCAAAGGCGGTAATACGGTTATCCACAGAATCAGGGGATAACG
CAGGAAAGAACATGTGAGCAAAAGGCCAGCAAAAGGCCAGGAACCGTA
AAAAGGCCGCGTTGCTGGCGTTTTTCCATAGGCTCCGCCCCCCTGACG
AGCATCACAAAAATCGACGCTCAAGTCAGAGGTGGCGAAACCCGACAG
GACTATAAAGATACCAGGCGTTTCCCCCTGGAAGCTCCCTCGTGCGCT
CTCCTGTTCCGACCCTGCCGCTTACCGGATACCTGTCCGCCTTTCTCC
CTTCGGGAAGCGTGGCGCTTTCTCAATGCTCACGCTGTAGGTATCTCA
GTTCGGTGTAGGTCGTTCGCTCCAAGCTGGGCTGTGTGCACGAACCCC
CCGTTCAGCCCGACCGCTGCGCCTTATCCGGTAACTATCGTCTTGAGT
CCAACCCGGTAAGACACGACTTATCGCCACTGGCAGCAGCCACTGGTA
ACAGGATTAGCAGAGCGAGGTATGTAGGCGGTGCTACAGAGTTCTTGA
AGTGGTGGCCTAACTACGGCTACACTAGAAGGACAGTATTTGGTATCT
GCGCTCTGCTGAAGCCAGTTACCTTCGGAAAAAGAGTTGGTAGCTCTT
GATCCGGCAAACAAACCACCGCTGGTAGCGGTGGTTTTTTTGTTTGCA
AGCAGCAGATTACGCGCAGAAAAAAAGGATCTCAAGAAGATCCTTTGA
TCTTTTCTACGGGGTCTGACGCTCAGTGGAACGAAAACTCACGTTAAG
GGATTTTGGTCATGAGATTATCAAAAAGGATCTTCACCTAGATCCTTT
TAAATTAAAAATGAAGTTTTAAATCAATCTAAAGTATATATGAGTAAA
CTTGGTCTGACAGTTACCAATGCTTAATCAGTGAGGCACCTATCTCAG
CGATCTGTCTATTTCGTTCATCCATAGTTGCCTGACTCCCCGTCGTGT
AGATAACTACGATACGGGAGGGCTTACCATCTGGCCCCAGTGCTGCAA
TGATACCGCGAGACCCACGCTCACCGGCTCCAGATTTATCAGCAATAA
ACCAGCCAGCCGGAAGGGCCGAGCGCAGAAGTGGTCCTGCAACTTTAT
CCGCCTCCATCCAGTCTATTAATTGTTGCCGGGAAGCTAGAGTAAGTA
GTTCGCCAGTTAATAGTTTGCGCAACGTTGTTGCCATTGCTACAGGCA
TCGTGGTGTCACGCTCGTCGTTTGGTATGGCTTCATTCAGCTCCGGTT
CCCAACGATCAAGGCGAGTTACATGATCCCCCATGTTGTGCAAAAAAG
CGGTTAGCTCCTTCGGTCCTCCGATCGTTGTCAGAAGTAAGTTGGCCG
CAGTGTTATCACTCATGGTTATGGCAGCACTGCATAATTCTCTTACTG
TCATGCCATCCGTAAGATGCTTTTCTGTGACTGGTGAGTACTCAACCA
AGTCATTCTGAGAATAGTGTATGCGGCGACCGAGTTGCTCTTGCCCGG
CGTCAATACGGGATAATACCGCGCCACATAGCAGAACTTTAAAAGTGC
TCATCATTGGAAAACGTTCTTCGGGGCGAAAACTCTCAAGGATCTTAC
CGCTGTTGAGATCCAGTTCGATGTAACCCACTCGTGCACCCAACTGAT
CTTCAGCATCTTTTACTTTCACCAGCGTTTCTGGGTGAGCAAAAACAG
GAAGGCAAAATGCCGCAAAAAAGGGAATAAGGGCGACACGGAAATGTT
GAATACTCATACTCTTCCTTTTTCAATATTATTGAAGCATTTATCAGG
GTTATTGTCTCATGAGCGGATACATATTTGAATGTATTTAGAAAAATA
AACAAATAGGGGTTCCGCGCACATTTCCCCGAAAAGTGCCACCTGACG
TCTAAGAAACCATTATTATCATGACATTAACCTATAAAAATAGGCGTA
TCACGAGGCCCTTTCGTCTCGCGCGTTTCGGTGATGACGGTGAAAACC
TCTGACACATGCAGCTCCCGGAGACGGTCACAGCTTGTCTGTAAGCGG
ATGCCGGGAGCAGACAAGCCCGTCAGGGCGCGTCAGCGGGTGTTGGCG
GGTGTCGGGGCTGGCTTAACTATGCGGCATCAGAGCAGATTGTACTGA
GAGTGCACCATATGCGGTGTGAAATACCGCACAGATGCGTAAGGAGAA
AATACCGCATCAGGAAATTGTAAACGTTAATATTTTGTTAAAATTCGC
GTTAAATTTTTGTTAAATCAGCTCATTTTTTAACCAATAGGCCGAAAT
CGGCAAAATCCCTTATAAATCAAAAGAATAGACCGAGATAGGGTTGAG
TGTTGTTCCAGTTTGGAACAAGAGTCCACTATTAAAGAACGTGGACTC
CAACGTCAAAGGGCGAAAAACCGTCTATCAGGGCGATGGCCCACTACG
TGAACCATCACCCTAATCAAGTTTTTTGGGGTCGAGGTGCCGTAAAGC
ACTAAATCGGAACCCTAAAGGGAGCCCCCGATTTAGAGCTTGACGGGG
AAAGCCGGCGAACGTGGCGAGAAAGGAAGGGAAGAAAGCGAAAGGAGC
GGGCGCTAGGGCGCTGGCAAGTGTAGCGGTCACGCTGCGCGTAACCAC
CACACCCGCCGCGCTTAATGCGCCGCTACAGGGCGCGTCGCGCCATTC
GCCATTCAGGCTACGCAACTGTTGGGAAGGGCGATCGGTGCGGGCCTC
TTCGCTATTACGCCAGGCTGC

### Transfection of HEK 293 Cells

HEK 293 cells were grown on poly-lysine-coated coverslips in growth medium. 1 μl of each virus was used for each well as follows: Control cells without virus, cells with AAV2-OTOF n-terminal portion (AAV2-OTOF-NT), cells with AAV2-OTOF c-terminal portion (AAV2-OTOF-CT) and cells with both viruses (AAV2-OTOF-NT and AAV2-OTOF-CT). The cells were stained with anti-OTOF antibody and mounted on glass slides.

### OTOF Knock-Out Mice

The OTOF knock-out mice used were generated in a previous study (see Roux et al. (2006) Otoferlin, defective in a human deafness form, is essential for exocytosis at the auditory ribbon synapse. Cell. 127:277-289). Briefly, two fragments containing the genomic sequences 5′ and 3′ to exons 14 and 15 of Otof were amplified by PCR. The 5 kb BamHI-XhoI-BssHII and 6 kb BssHII-SfiI-BamHI-NaeI 129/SvPas fragments were inserted into pUC19 (New England BioLabs) previously modified by inserting a BamHI-XhoI-BssHII-SfiI-NaeI-HindIII polylinker. A loxP-hygro-loxP (the gene conferring resistance to hygromycin under control of the phosphoglycerate kinase gene [Pgk-1] promoter) cassette was inserted into the BssHII site. All constructs were sequenced, and the sequences obtained were compared with the 129/SvPas genomic sequence 282 CK35 ES cells resistant to hygromycin were screened for homologous recombination and monoinsertion events by Southern blot analysis. Two clones were injected into C57BL/6N blastocysts to create chimeric animals. Transmission of the mutant Otof allele was detected by PCR in agouti pups. Positive pups in the F1 progeny were crossed with Pgk-1-cre mice in a mixed C57BL/6-129/SvPas background. F2 animals carrying an allele in which the hygromycin selection cassette was deleted (Otof tm1Ugds allele) were selected by PCR, using primers 5′-CACTTGCTTTGTCT CATCTCC-3′ (SEQ ID NO: 12) and 5′-GTCACTTCTTCTGGGTATTTC-3′ (SEQ ID NO: 13), generating a 507 base pair PCR product. The heterozygous animals were interbred to generate Otof^{-/-}, Otof^{+/-}, and Otof^{+/+} mice. The knockout mice were generated in C57BL/6-129/SvPas background as described above. Because this background strain is known to have some age-related hearing loss, the mice were backcrossed with FVB mice strain to the 10^{th} generation to obtain an FVB homogeneous genetic background with no known age-related hearing loss.

### Delivery of AAV to Mice

OTOF knock-out mice (newborn and older than P10) were injected with 1 microliter of viral particles of each of the two AAV constructs using a round window membrane (RWM) injection as previously described (see Akil et al. (2012) Restoration of Hearing in the VGLUT3 Knockout Mouse Using Virally-Mediated Gene Therapy. Neuron. 75(2): 283-293 and Akil et al. (2015) Surgical Method for Virally Mediated Gene Delivery to the Mouse Inner Ear through the Round Window Membrane. J. Vis. Exp. (97), e52187). AAV2-OTOF-NT (6.32×10¹²vg/ml) and AAV2-OTOF-CT (4.5×10¹² vg/ml) were delivered through the RWM to P1-3 mice. AAV2-OTOF-NT (1.43×10¹³ vg/ml) and AAV2-OTOF-CT (3.12×10¹³vg/ml) were delivered through the RWM to P≥12 mice. ABR tests were conducted 7 days after injection. Expression of OTOF protein in the mice was measured using anti-OTOF antibody to label cells in cochlear whole mounts. Reverse-transcriptase (RT)-PCR was used to screen for the presence of OTOF mRNA within the cochlear tissue of the mice. Wild-type mice were also injected using the same technique with AAV2-GFP to assess viral delivery to cochlea with AAV2 serotype. Cochlea were whole mounted and stained with anti-GFP antibody.

### Auditory Brainstem Response (ABR) Testing

Hearing tests were performed as previously described (Akil et al. (2006) Progressive deafness and altered cochlear innervation in knockout mice lacking prosaposin. J. Neurosci. 26:13076-13088 and Akil et al. (2016) Mouse Auditory Brainstem Response Testing. Bio Protoc. 6 (6)) with the otoferlin knockout (OTOF KO) mice, rescued OTOF KO mice and wild-type (WT) littermates. Briefly, all auditory testing was performed in a sound-proof chamber. Before acoustic testing, mice were anesthetized by intraperitoneal injection of a mixture of ketamine hydrochloride (Ketaset, 100 mg/ml) and xylazine hydrochloride (xyla-ject, 10 mg/ml) and boosted with one-fifth the original dose as required. Body temperature was maintained with a heating pad and monitored with a rectal probe throughout recording.

The evoked acoustic brainstem response (ABR) thresholds were differentially recorded from the scalp of the mice. Responses were recorded using subdermal needle electrodes at the vertex, below the pinna of the left ear (reference), and below the contralateral ear (ground). The sound stimuli used included clicks (5 ms duration, 31 Hz) and tone pips at 8, 16, and 32 kHz (10 ms duration, cos2 shaping, 21 Hz). Measurements were recorded using the TDT BioSig III system (Tucker Davis Technologies). For each stimulus, electroencephalographic (EEG) activity was recorded for 20 ms (at a sampling rate of 25 kHz) and filtered (0.3-3 kHz). Waveforms from 512 stimuli were averaged for click responses. Waveforms from 1000 stimuli were examined to identify frequency-specific tone-burst stimuli (8, 16, and 32 kHz). ABR waveforms were recorded in 5 dB sound pressure level (SPL) intervals down from the maximum amplitude. The threshold was defined as the lowest stimulus level at which response peaks for waves I-V were clearly and repetitively present upon visual inspection. These threshold judgments were confirmed by analysis of stored waveforms. The comparison of each group of animals was performed using one way ANOVA with Bonferroni's post hoc testing.

### Results

Two different AAV plasmid constructs were generated to deliver the 5′ half and the 3′ half of the mouse OTOF cDNA to the inner ear of OTOF knock-out mice (OTOF N-terminal virus and OTOF C-terminal virus). The two constructs were packaged separately into AAV2 particles. The AAV2 particles were then pooled together and used to treat HEK 293 cells or were injected into the inner ear of OTOF knock-out mice.

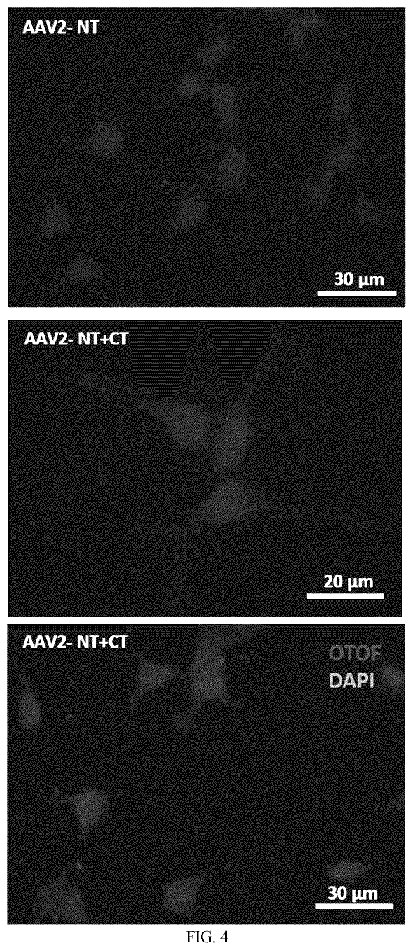

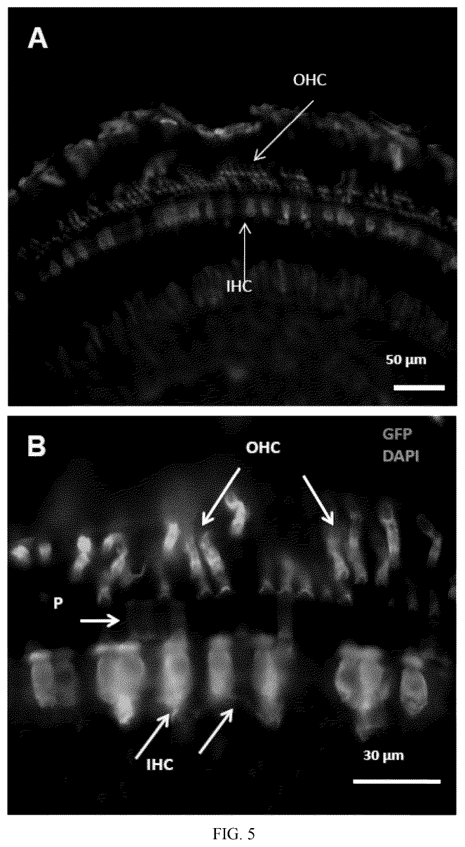

It was shown that HEK 293 cells only expressed Otoferlin protein when transfected with both viruses (FIG. 4). No expression of Otoferlin protein was observed in untreated cells, or in cells transfected with only the OTOF N-terminal (FIG. 4) or OTOF C-terminal virus.

Next, the ability of AAV2 to transduce the mouse cochlea was assess using an AAV2-GFP reporter virus. It was shown that AAV2 transfects a number of cell types including inner hair cells (IHC), outer hair cells (OHC), pillar cells (P) and other supporting cells (SC) in the organ of Corti (FIG. 5). Thus, AAV2 can transduce mouse cochlea effectively.

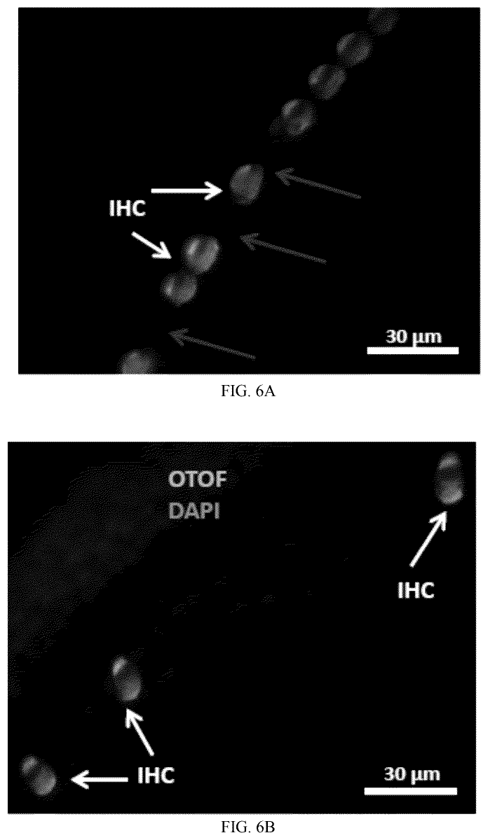

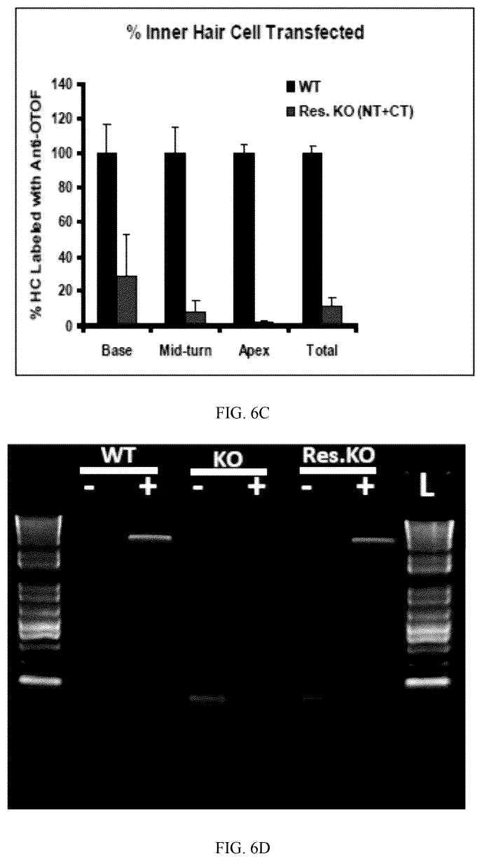

Mice were then treated with the pooled OTOF N-terminal and C-terminal viruses and compared to various controls. Otoferlin protein was found to be expressed upon treatment with both viruses (FIG. 6). The largest number of transfected inner hair cells (IHCs) were observed in the base and fewer in the mid-turn and apex (FIG. 6). IHCs counts demonstrated that ˜11% of IHCs were labeled overall, with significant differences seen between the base (˜29%), mid-turn (˜8%), and apex (˜2%). (FIG. 6). RT-PCR was used to show that OTOF mRNA was in whole cochlear extract and was the same size in both wild-type and OTOF knock-out mice treated with both viruses (FIG. 6). In contrast, the untreated OTOF knock-out mouse cochleae did not demonstrate OTOF mRNA expression. No products were detected when RT-PCR was performed in the absence of reverse transcriptase.

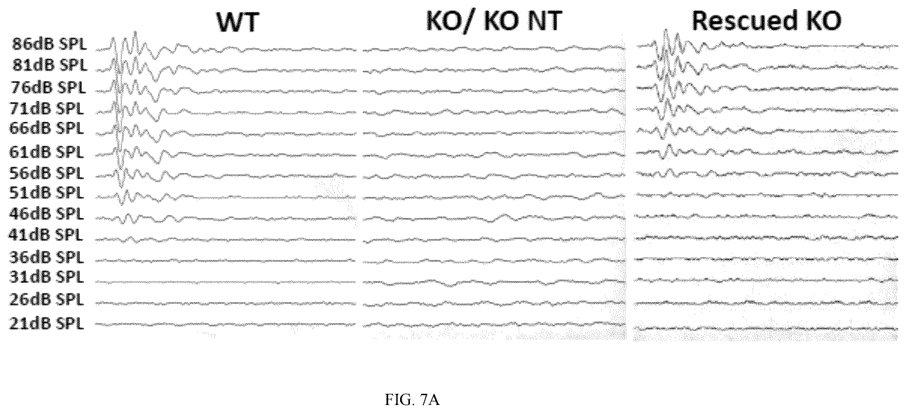

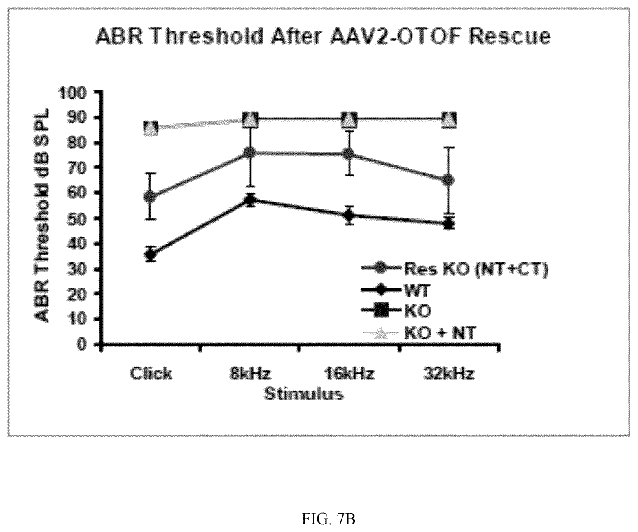

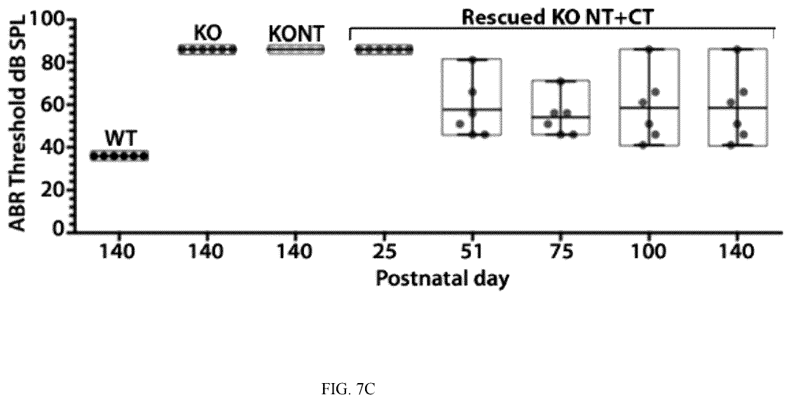

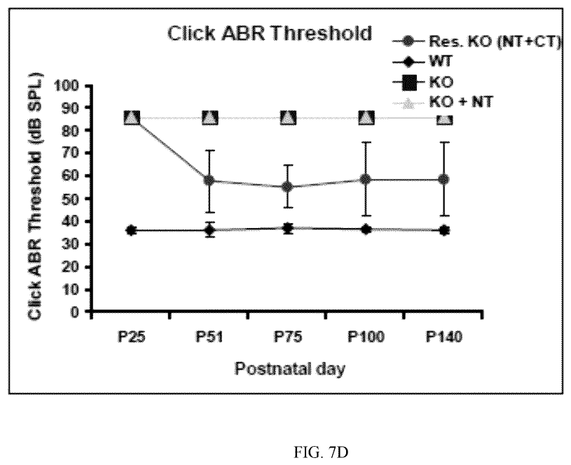

Next, hearing tests were performed to determine whether the Otoferlin expressed by the delivery of both the N-and C-terminal viruses was capable of rescuing hearing function. ABR waveforms from the wild-type and the OTOF knock-out mice treated with both viruses were similar, documenting hearing recovery in the rescued KO mice whereas untreated OTOF knock-out mice controls and the OTOF knock-out mice transfected with just OTOF N-terminal virus show no hearing recovery (FIG. 7). At P70, partial hearing recovery (improved ABR thresholds) was seen to clicks and at specific frequencies 8, 16 and 32 kHz in the OTOF knock-out mice treated with both viruses, while at 8 and 16 kHz the ABR thresholds appear to be slightly elevated, though still significantly better than untreated OTOF knock-out mice (FIG. 7). Remarkably, hearing was maintained in the OTOF knock-out mice treated with both viruses (KO NT+CT) for more than 4 months (FIG. 7), although ABR thresholds were somewhat variable. Non-transfected KO controls and the KO transfected with OTOF NT remained deaf (FIG. 7).

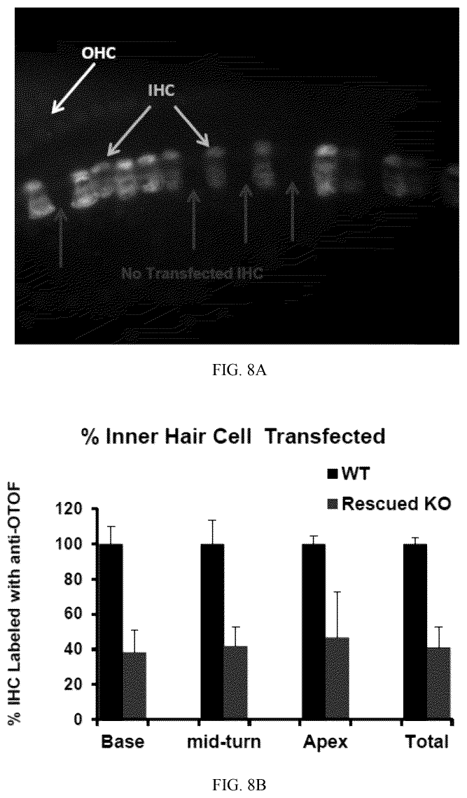

Next, OTOF knock-out mice older than P12 treated with both viruses. The dually transfected IHCs expressed OTOF, with homogenous transfection rates seen in the base (not shown) and the apex (FIG. 8). IHCs counts demonstrated that ˜41% of IHCs were labeled overall, with slight differences seen between the base (˜38%), mid-turn (˜42%), and apex (˜47%) (FIG. 8).

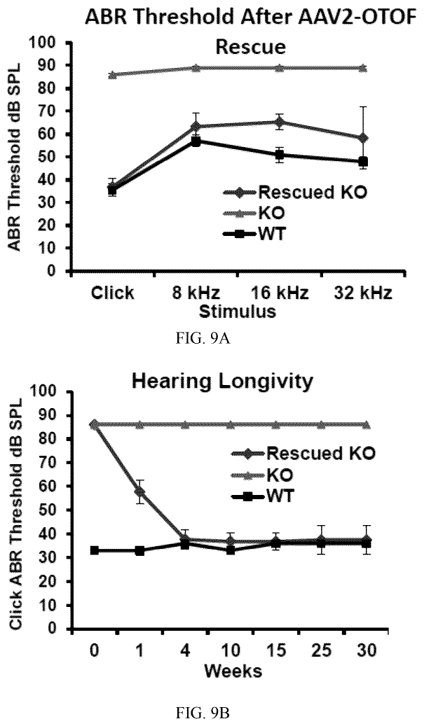

At P60 all OTOF knock-out mice treated with both viruses demonstrated normal ABR threshold to clicks stimulus while at specific frequencies 8, 16 and 32 kHz the ABR thresholds appeared to be slightly elevated, though still significantly better than untreated OTOF knock-out mice (FIG. 9). A time course of hearing recovery following injection of both viruses into OTOF knock-out mice at an age older than P12 showed that hearing was maintained in the treated mice for more than 30 weeks, and the ABR thresholds were restored to the WT levels (FIG. 9).

These results demonstrate that a use of more than one AAV construct to deliver different parts of an OTOF cDNA can result in a functional cDNA in vivo. These results also demonstrate that hearing loss can be treated by delivery of OTOF cDNA using an AAV delivery system.

### Example 2: Human OTOF Dual Vector Constructs

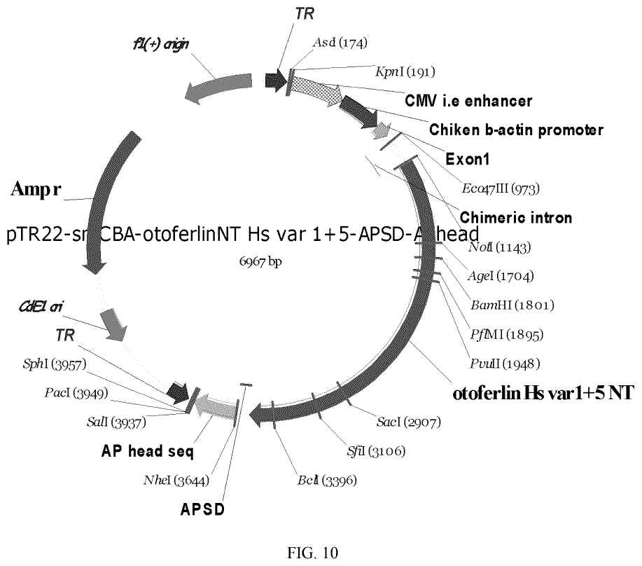

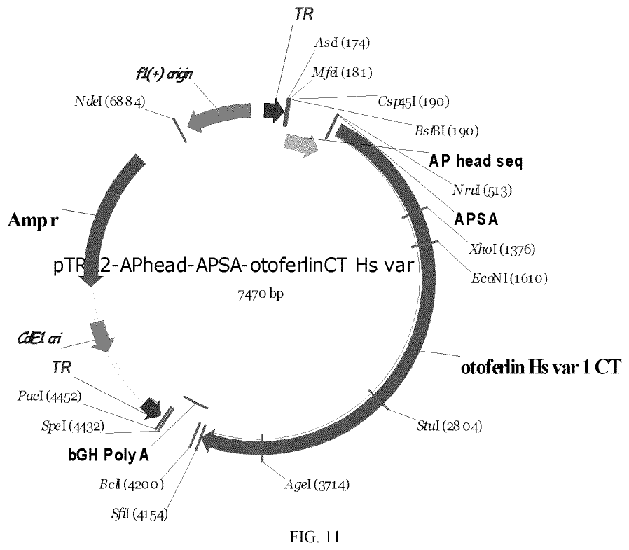

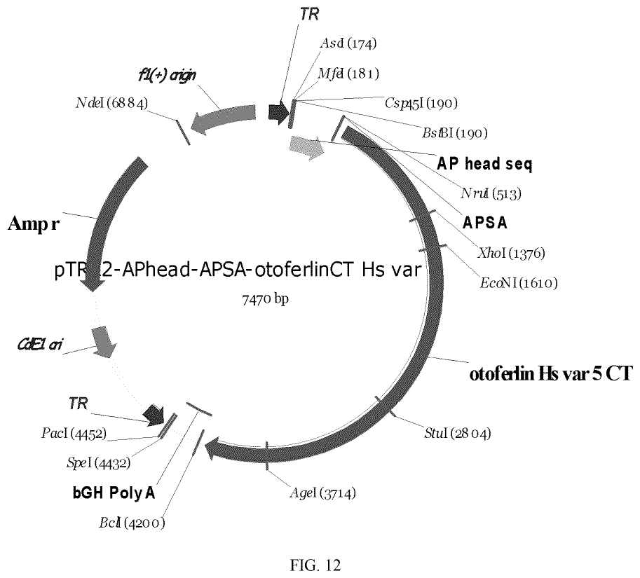

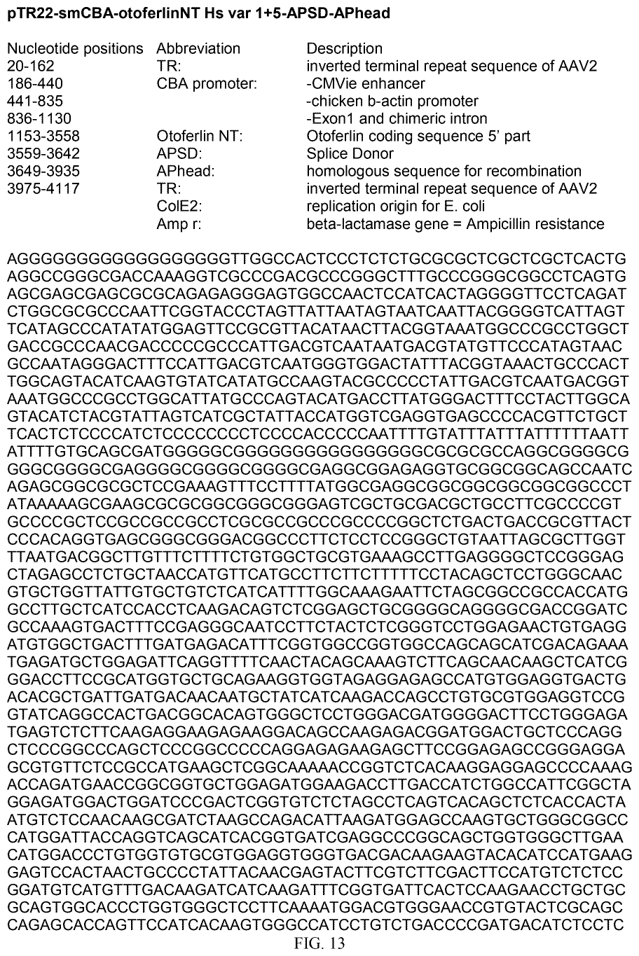

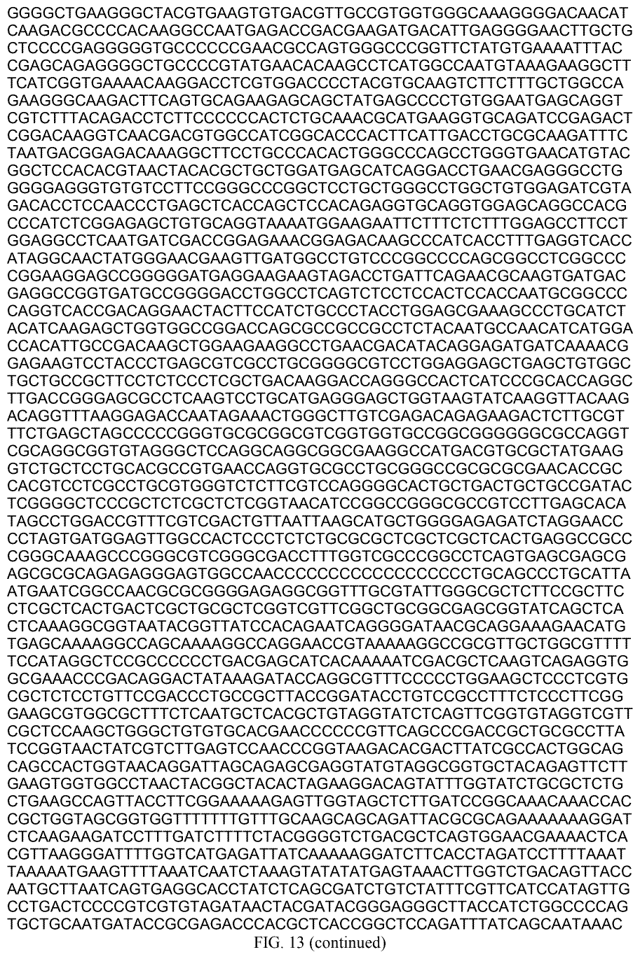

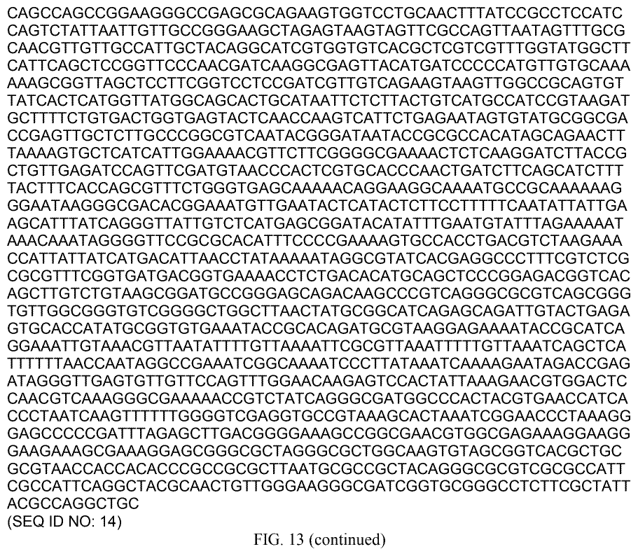

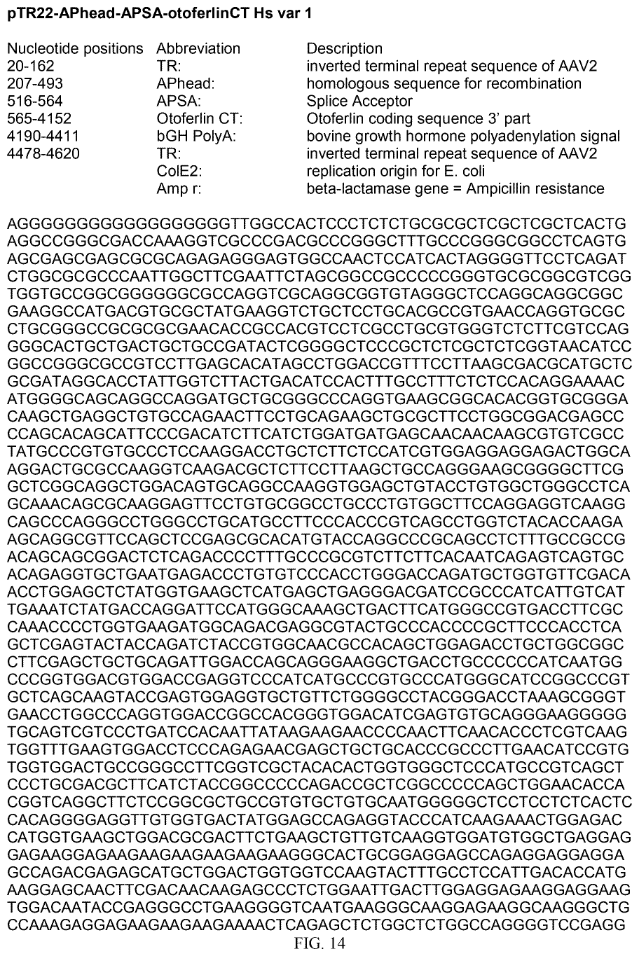

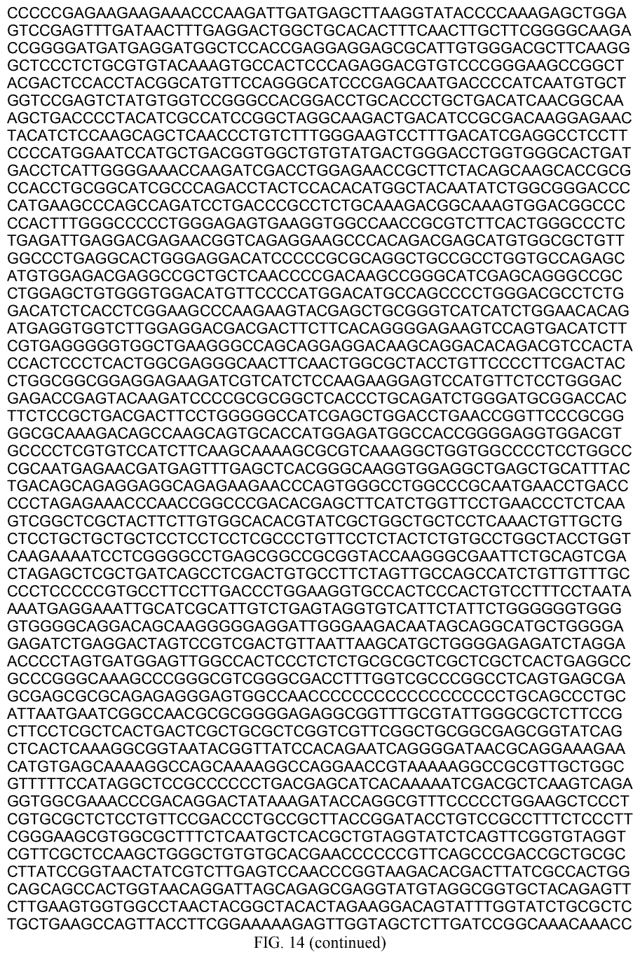

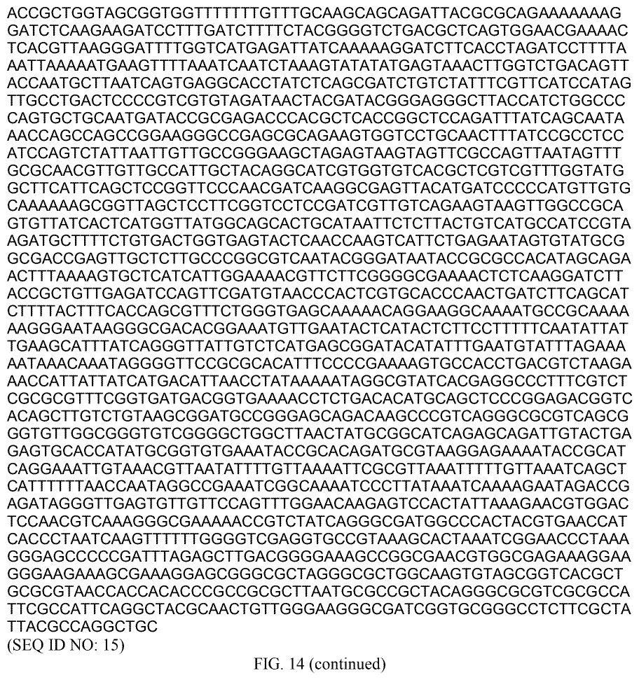

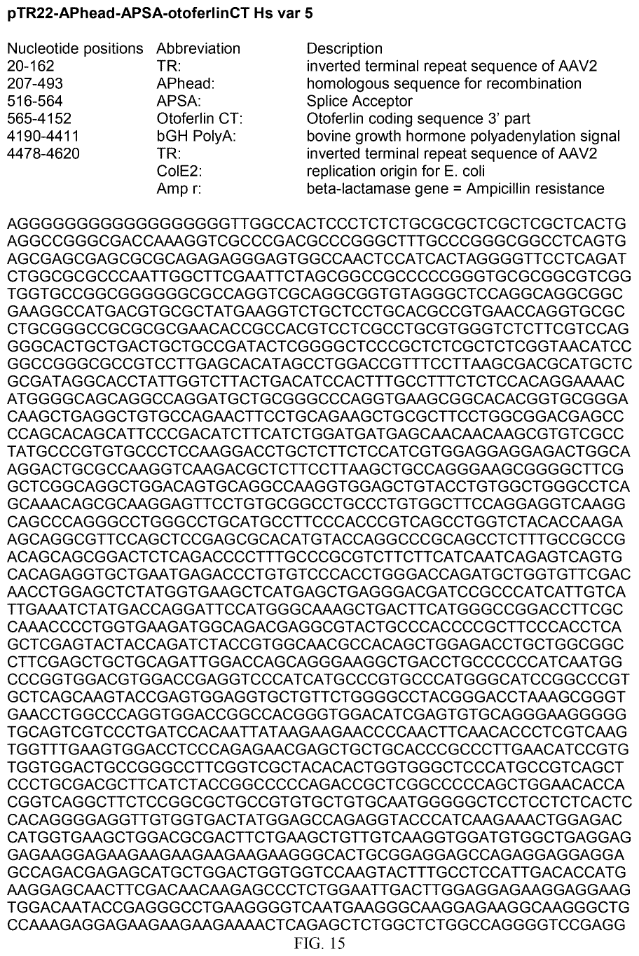

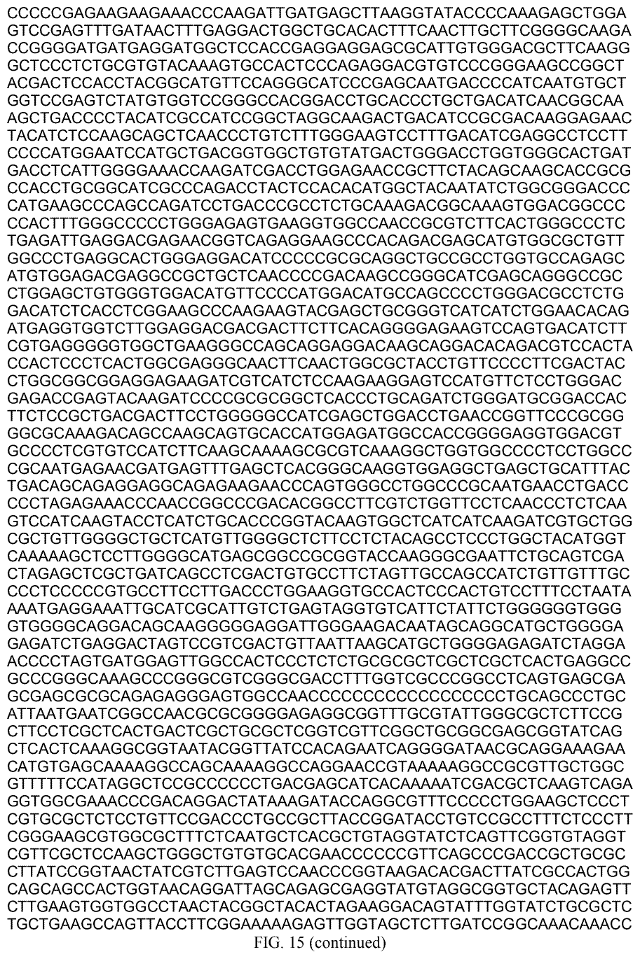

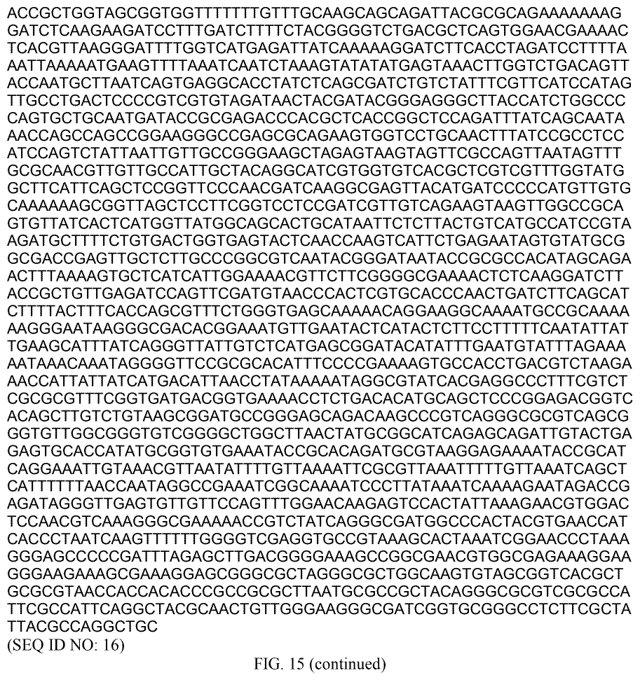

Provided below are example dual vector sequences for expressing Human Otoferlin protein isoforms 1 and 5. The cDNAs encoding both isoforms 1 and 5 contain the same N-terminal sequence such that the same N-terminal vector can be used for expressing both isoforms. Vector maps and annotated sequences corresponding to the below sequences are shown in FIGS. 10-15.

pTR22-smCBA-otoferlinNT Hs var 1 + 5-APSD-APhead
(SEQ ID NO: 14)
AGGGGGGGGGGGGGGGGGGTTGGCCACTCCCTCTCTGCGCGCTCGCTC
GCTCACTGAGGCCGGGCGACCAAAGGTCGCCCGACGCCCGGGCTTTGC
CCGGGCGGCCTCAGTGAGCGAGCGAGCGCGCAGAGAGGGAGTGGCCAA
CTCCATCACTAGGGGTTCCTCAGATCTGGCGCGCCCAATTCGGTACCC
TAGTTATTAATAGTAATCAATTACGGGGTCATTAGTTCATAGCCCATA
TATGGAGTTCCGCGTTACATAACTTACGGTAAATGGCCCGCCTGGCTG
ACCGCCCAACGACCCCCGCCCATTGACGTCAATAATGACGTATGTTCC
CATAGTAACGCCAATAGGGACTTTCCATTGACGTCAATGGGTGGACTA
TTTACGGTAAACTGCCCACTTGGCAGTACATCAAGTGTATCATATGCC
AAGTACGCCCCCTATTGACGTCAATGACGGTAAATGGCCCGCCTGGCA
TTATGCCCAGTACATGACCTTATGGGACTTTCCTACTTGGCAGTACAT
CTACGTATTAGTCATCGCTATTACCATGGTCGAGGTGAGCCCCACGTT
CTGCTTCACTCTCCCCATCTCCCCCCCCTCCCCACCCCCAATTTTGTA
TTTATTTATTTTTTAATTATTTTGTGCAGCGATGGGGGCGGGGGGGGG
GGGGGGGCGCGCGCCAGGCGGGGCGGGGCGGGGCGAGGGGCGGGGCGG
GGCGAGGCGGAGAGGTGCGGCGGCAGCCAATCAGAGCGGCGCGCTCCG
AAAGTTTCCTTTTATGGCGAGGCGGCGGCGGCGGCGGCCCTATAAAAA
GCGAAGCGCGCGGCGGGCGGGAGTCGCTGCGACGCTGCCTTCGCCCCG
TGCCCCGCTCCGCCGCCGCCTCGCGCCGCCCGCCCCGGCTCTGACTGA
CCGCGTTACTCCCACAGGTGAGCGGGCGGGACGGCCCTTCTCCTCCGG
GCTGTAATTAGCGCTTGGTTTAATGACGGCTTGTTTCTTTTCTGTGGC
TGCGTGAAAGCCTTGAGGGGCTCCGGGAGCTAGAGCCTCTGCTAACCA
TGTTCATGCCTTCTTCTTTTTCCTACAGCTCCTGGGCAACGTGCTGGT
TATTGTGCTGTCTCATCATTTTGGCAAAGAATTCTAGCGGCCGCCACC
ATGGCCTTGCTCATCCACCTCAAGACAGTCTCGGAGCTGCGGGGCAGG
GGCGACCGGATCGCCAAAGTGACTTTCCGAGGGCAATCCTTCTACTCT
CGGGTCCTGGAGAACTGTGAGGATGTGGCTGACTTTGATGAGACATTT
CGGTGGCCGGTGGCCAGCAGCATCGACAGAAATGAGATGCTGGAGATT
CAGGTTTTCAACTACAGCAAAGTCTTCAGCAACAAGCTCATCGGGACC
TTCCGCATGGTGCTGCAGAAGGTGGTAGAGGAGAGCCATGTGGAGGTG
ACTGACACGCTGATTGATGACAACAATGCTATCATCAAGACCAGCCTG
TGCGTGGAGGTCCGGTATCAGGCCACTGACGGCACAGTGGGCTCCTGG
GACGATGGGGACTTCCTGGGAGATGAGTCTCTTCAAGAGGAAGAGAAG
GACAGCCAAGAGACGGATGGACTGCTCCCAGGCTCCCGGCCCAGCTCC
CGGCCCCCAGGAGAGAAGAGCTTCCGGAGAGCCGGGAGGAGCGTGTTC
TCCGCCATGAAGCTCGGCAAAAACCGGTCTCACAAGGAGGAGCCCCAA
AGACCAGATGAACCGGCGGTGCTGGAGATGGAAGACCTTGACCATCTG
GCCATTCGGCTAGGAGATGGACTGGATCCCGACTCGGTGTCTCTAGCC
TCAGTCACAGCTCTCACCACTAATGTCTCCAACAAGCGATCTAAGCCA
GACATTAAGATGGAGCCAAGTGCTGGGCGGCCCATGGATTACCAGGTC
AGCATCACGGTGATCGAGGCCCGGCAGCTGGTGGGCTTGAACATGGAC
CCTGTGGTGTGCGTGGAGGTGGGTGACGACAAGAAGTACACATCCATG
AAGGAGTCCACTAACTGCCCCTATTACAACGAGTACTTCGTCTTCGAC
TTCCATGTCTCTCCGGATGTCATGTTTGACAAGATCATCAAGATTTCG
GTGATTCACTCCAAGAACCTGCTGCGCAGTGGCACCCTGGTGGGCTCC
TTCAAAATGGACGTGGGAACCGTGTACTCGCAGCCAGAGCACCAGTTC
CATCACAAGTGGGCCATCCTGTCTGACCCCGATGACATCTCCTCGGGG
CTGAAGGGCTACGTGAAGTGTGACGTTGCCGTGGTGGGCAAAGGGGAC
AACATCAAGACGCCCCACAAGGCCAATGAGACCGACGAAGATGACATT
GAGGGGAACTTGCTGCTCCCCGAGGGGGTGCCCCCCGAACGCCAGTGG
GCCCGGTTCTATGTGAAAATTTACCGAGCAGAGGGGCTGCCCCGTATG
AACACAAGCCTCATGGCCAATGTAAAGAAGGCTTTCATCGGTGAAAAC
AAGGACCTCGTGGACCCCTACGTGCAAGTCTTCTTTGCTGGCCAGAAG
GGCAAGACTTCAGTGCAGAAGAGCAGCTATGAGCCCCTGTGGAATGAG
CAGGTCGTCTTTACAGACCTCTTCCCCCCACTCTGCAAACGCATGAAG
GTGCAGATCCGAGACTCGGACAAGGTCAACGACGTGGCCATCGGCACC
CACTTCATTGACCTGCGCAAGATTTCTAATGACGGAGACAAAGGCTTC
CTGCCCACACTGGGCCCAGCCTGGGTGAACATGTACGGCTCCACACGT
AACTACACGCTGCTGGATGAGCATCAGGACCTGAACGAGGGCCTGGGG
GAGGGTGTGTCCTTCCGGGCCCGGCTCCTGCTGGGCCTGGCTGTGGAG
ATCGTAGACACCTCCAACCCTGAGCTCACCAGCTCCACAGAGGTGCAG
GTGGAGCAGGCCACGCCCATCTCGGAGAGCTGTGCAGGTAAAATGGAA
GAATTCTTTCTCTTTGGAGCCTTCCTGGAGGCCTCAATGATCGACCGG
AGAAACGGAGACAAGCCCATCACCTTTGAGGTCACCATAGGCAACTAT
GGGAACGAAGTTGATGGCCTGTCCCGGCCCCAGCGGCCTCGGCCCCGG
AAGGAGCCGGGGGATGAGGAAGAAGTAGACCTGATTCAGAACGCAAGT
GATGACGAGGCCGGTGATGCCGGGGACCTGGCCTCAGTCTCCTCCACT
CCACCAATGCGGCCCCAGGTCACCGACAGGAACTACTTCCATCTGCCC
TACCTGGAGCGAAAGCCCTGCATCTACATCAAGAGCTGGTGGCCGGAC
CAGCGCCGCCGCCTCTACAATGCCAACATCATGGACCACATTGCCGAC
AAGCTGGAAGAAGGCCTGAACGACATACAGGAGATGATCAAAACGGAG
AAGTCCTACCCTGAGCGTCGCCTGCGGGGCGTCCTGGAGGAGCTGAGC
TGTGGCTGCTGCCGCTTCCTCTCCCTCGCTGACAAGGACCAGGGCCAC
TCATCCCGCACCAGGCTTGACCGGGAGCGCCTCAAGTCCTGCATGAGG
GAGCTGGTAAGTATCAAGGTTACAAGACAGGTTTAAGGAGACCAATAG
AAACTGGGCTTGTCGAGACAGAGAAGACTCTTGCGTTTCTGAGCTAGC
CCCCGGGTGCGCGGCGTCGGTGGTGCCGGCGGGGGGCGCCAGGTCGCA
GGCGGTGTAGGGCTCCAGGCAGGCGGCGAAGGCCATGACGTGCGCTAT
GAAGGTCTGCTCCTGCACGCCGTGAACCAGGTGCGCCTGCGGGCCGCG
CGCGAACACCGCCACGTCCTCGCCTGCGTGGGTCTCTTCGTCCAGGGG
CACTGCTGACTGCTGCCGATACTCGGGGCTCCCGCTCTCGCTCTCGGT
AACATCCGGCCGGGCGCCGTCCTTGAGCACATAGCCTGGACCGTTTCG
TCGACTGTTAATTAAGCATGCTGGGGAGAGATCTAGGAACCCCTAGTG
ATGGAGTTGGCCACTCCCTCTCTGCGCGCTCGCTCGCTCACTGAGGCC
GCCCGGGCAAAGCCCGGGCGTCGGGCGACCTTTGGTCGCCCGGCCTCA
GTGAGCGAGCGAGCGCGCAGAGAGGGAGTGGCCAACCCCCCCCCCCCC
CCCCCTGCAGCCCTGCATTAATGAATCGGCCAACGCGCGGGGAGAGGC
GGTTTGCGTATTGGGCGCTCTTCCGCTTCCTCGCTCACTGACTCGCTG
CGCTCGGTCGTTCGGCTGCGGCGAGCGGTATCAGCTCACTCAAAGGCG
GTAATACGGTTATCCACAGAATCAGGGGATAACGCAGGAAAGAACATG
TGAGCAAAAGGCCAGCAAAAGGCCAGGAACCGTAAAAAGGCCGCGTTG
CTGGCGTTTTTCCATAGGCTCCGCCCCCCTGACGAGCATCACAAAAAT
CGACGCTCAAGTCAGAGGTGGCGAAACCCGACAGGACTATAAAGATAC
CAGGCGTTTCCCCCTGGAAGCTCCCTCGTGCGCTCTCCTGTTCCGACC
CTGCCGCTTACCGGATACCTGTCCGCCTTTCTCCCTTCGGGAAGCGTG
GCGCTTTCTCAATGCTCACGCTGTAGGTATCTCAGTTCGGTGTAGGTC
GTTCGCTCCAAGCTGGGCTGTGTGCACGAACCCCCCGTTCAGCCCGAC
CGCTGCGCCTTATCCGGTAACTATCGTCTTGAGTCCAACCCGGTAAGA
CACGACTTATCGCCACTGGCAGCAGCCACTGGTAACAGGATTAGCAGA
GCGAGGTATGTAGGCGGTGCTACAGAGTTCTTGAAGTGGTGGCCTAAC
TACGGCTACACTAGAAGGACAGTATTTGGTATCTGCGCTCTGCTGAAG
CCAGTTACCTTCGGAAAAAGAGTTGGTAGCTCTTGATCCGGCAAACAA
ACCACCGCTGGTAGCGGTGGTTTTTTTGTTTGCAAGCAGCAGATTACG
CGCAGAAAAAAAGGATCTCAAGAAGATCCTTTGATCTTTTCTACGGGG
TCTGACGCTCAGTGGAACGAAAACTCACGTTAAGGGATTTTGGTCATG
AGATTATCAAAAAGGATCTTCACCTAGATCCTTTTAAATTAAAAATGA
AGTTTTAAATCAATCTAAAGTATATATGAGTAAACTTGGTCTGACAGT
TACCAATGCTTAATCAGTGAGGCACCTATCTCAGCGATCTGTCTATTT
CGTTCATCCATAGTTGCCTGACTCCCCGTCGTGTAGATAACTACGATA
CGGGAGGGCTTACCATCTGGCCCCAGTGCTGCAATGATACCGCGAGAC
CCACGCTCACCGGCTCCAGATTTATCAGCAATAAACCAGCCAGCCGGA
AGGGCCGAGCGCAGAAGTGGTCCTGCAACTTTATCCGCCTCCATCCAG
TCTATTAATTGTTGCCGGGAAGCTAGAGTAAGTAGTTCGCCAGTTAAT
AGTTTGCGCAACGTTGTTGCCATTGCTACAGGCATCGTGGTGTCACGC
TCGTCGTTTGGTATGGCTTCATTCAGCTCCGGTTCCCAACGATCAAGG
CGAGTTACATGATCCCCCATGTTGTGCAAAAAAGCGGTTAGCTCCTTC
GGTCCTCCGATCGTTGTCAGAAGTAAGTTGGCCGCAGTGTTATCACTC
ATGGTTATGGCAGCACTGCATAATTCTCTTACTGTCATGCCATCCGTA
AGATGCTTTTCTGTGACTGGTGAGTACTCAACCAAGTCATTCTGAGAA
TAGTGTATGCGGCGACCGAGTTGCTCTTGCCCGGCGTCAATACGGGAT
AATACCGCGCCACATAGCAGAACTTTAAAAGTGCTCATCATTGGAAAA
CGTTCTTCGGGGCGAAAACTCTCAAGGATCTTACCGCTGTTGAGATCC
AGTTCGATGTAACCCACTCGTGCACCCAACTGATCTTCAGCATCTTTT
ACTTTCACCAGCGTTTCTGGGTGAGCAAAAACAGGAAGGCAAAATGCC
GCAAAAAAGGGAATAAGGGCGACACGGAAATGTTGAATACTCATACTC
TTCCTTTTTCAATATTATTGAAGCATTTATCAGGGTTATTGTCTCATG
AGCGGATACATATTTGAATGTATTTAGAAAAATAAACAAATAGGGGTT
CCGCGCACATTTCCCCGAAAAGTGCCACCTGACGTCTAAGAAACCATT
ATTATCATGACATTAACCTATAAAAATAGGCGTATCACGAGGCCCTTT
CGTCTCGCGCGTTTCGGTGATGACGGTGAAAACCTCTGACACATGCAG
CTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGG
CTTAACTATGCGGCATCAGAGCAGATTGTACTGAGAGTGCACCATATG
CGGTGTGAAATACCGCACAGATGCGTAAGGAGAAAATACCGCATCAGG
AAATTGTAAACGTTAATATTTTGTTAAAATTCGCGTTAAATTTTTGTT
AAATCAGCTCATTTTTTAACCAATAGGCCGAAATCGGCAAAATCCCTT
ATAAATCAAAAGAATAGACCGAGATAGGGTTGAGTGTTGTTCCAGTTT
GGAACAAGAGTCCACTATTAAAGAACGTGGACTCCAACGTCAAAGGGC
GAAAAACCGTCTATCAGGGCGATGGCCCACTACGTGAACCATCACCCT
AATCAAGTTTTTTGGGGTCGAGGTGCCGTAAAGCACTAAATCGGAACC
CTAAAGGGAGCCCCCGATTTAGAGCTTGACGGGGAAAGCCGGCGAACG
TGGCGAGAAAGGAAGGGAAGAAAGCGAAAGGAGCGGGCGCTAGGGCGC
TGGCAAGTGTAGCGGTCACGCTGCGCGTAACCACCACACCCGCCGCGC
TTAATGCGCCGCTACAGGGCGCGTCGCGCCATTCGCCATTCAGGCTAC
GCAACTGTTGGGAAGGGCGATCGGTGCGGGCCTCTTCGCTATTACGCC
AGGCTGC
pTR22-APhead-APSA-otoferlinCT Hs var 1
(SEQ ID NO: 15)
AGGGGGGGGGGGGGGGGGGTTGGCCACTCCCTCTCTGCGCGCTCGCTC
GCTCACTGAGGCCGGGCGACCAAAGGTCGCCCGACGCCCGGGCTTTGC
CCGGGCGGCCTCAGTGAGCGAGCGAGCGCGCAGAGAGGGAGTGGCCAA
CTCCATCACTAGGGGTTCCTCAGATCTGGCGCGCCCAATTGGCTTCGA
ATTCTAGCGGCCGCCCCCGGGTGCGCGGCGTCGGTGGTGCCGGCGGGG
GGCGCCAGGTCGCAGGCGGTGTAGGGCTCCAGGCAGGCGGCGAAGGCC
ATGACGTGCGCTATGAAGGTCTGCTCCTGCACGCCGTGAACCAGGTGC
GCCTGCGGGCCGCGCGCGAACACCGCCACGTCCTCGCCTGCGTGGGTC
TCTTCGTCCAGGGGCACTGCTGACTGCTGCCGATACTCGGGGCTCCCG
CTCTCGCTCTCGGTAACATCCGGCCGGGCGCCGTCCTTGAGCACATAG
CCTGGACCGTTTCCTTAAGCGACGCATGCTCGCGATAGGCACCTATTG
GTCTTACTGACATCCACTTTGCCTTTCTCTCCACAGGAAAACATGGGG
CAGCAGGCCAGGATGCTGCGGGCCCAGGTGAAGCGGCACACGGTGCGG
GACAAGCTGAGGCTGTGCCAGAACTTCCTGCAGAAGCTGCGCTTCCTG
GCGGACGAGCCCCAGCACAGCATTCCCGACATCTTCATCTGGATGATG
AGCAACAACAAGCGTGTCGCCTATGCCCGTGTGCCCTCCAAGGACCTG
CTCTTCTCCATCGTGGAGGAGGAGACTGGCAAGGACTGCGCCAAGGTC
AAGACGCTCTTCCTTAAGCTGCCAGGGAAGCGGGGCTTCGGCTCGGCA
GGCTGGACAGTGCAGGCCAAGGTGGAGCTGTACCTGTGGCTGGGCCTC
AGCAAACAGCGCAAGGAGTTCCTGTGCGGCCTGCCCTGTGGCTTCCAG
GAGGTCAAGGCAGCCCAGGGCCTGGGCCTGCATGCCTTCCCACCCGTC
AGCCTGGTCTACACCAAGAAGCAGGCGTTCCAGCTCCGAGCGCACATG
TACCAGGCCCGCAGCCTCTTTGCCGCCGACAGCAGCGGACTCTCAGAC
CCCTTTGCCCGCGTCTTCTTCATCAATCAGAGTCAGTGCACAGAGGTG
CTGAATGAGACCCTGTGTCCCACCTGGGACCAGATGCTGGTGTTCGAC
AACCTGGAGCTCTATGGTGAAGCTCATGAGCTGAGGGACGATCCGCCC
ATCATTGTCATTGAAATCTATGACCAGGATTCCATGGGCAAAGCTGAC
TTCATGGGCCGGACCTTCGCCAAACCCCTGGTGAAGATGGCAGACGAG
GCGTACTGCCCACCCCGCTTCCCACCTCAGCTCGAGTACTACCAGATC
TACCGTGGCAACGCCACAGCTGGAGACCTGCTGGCGGCCTTCGAGCTG
CTGCAGATTGGACCAGCAGGGAAGGCTGACCTGCCCCCCATCAATGGC
CCGGTGGACGTGGACCGAGGTCCCATCATGCCCGTGCCCATGGGCATC
CGGCCCGTGCTCAGCAAGTACCGAGTGGAGGTGCTGTTCTGGGGCCTA
CGGGACCTAAAGCGGGTGAACCTGGCCCAGGTGGACCGGCCACGGGTG
GACATCGAGTGTGCAGGGAAGGGGGTGCAGTCGTCCCTGATCCACAAT
TATAAGAAGAACCCCAACTTCAACACCCTCGTCAAGTGGTTTGAAGTG
GACCTCCCAGAGAACGAGCTGCTGCACCCGCCCTTGAACATCCGTGTG
GTGGACTGCCGGGCCTTCGGTCGCTACACACTGGTGGGCTCCCATGCC
GTCAGCTCCCTGCGACGCTTCATCTACCGGCCCCCAGACCGCTCGGCC
CCCAGCTGGAACACCACGGTCAGGCTTCTCCGGCGCTGCCGTGTGCTG
TGCAATGGGGGCTCCTCCTCTCACTCCACAGGGGAGGTTGTGGTGACT
ATGGAGCCAGAGGTACCCATCAAGAAACTGGAGACCATGGTGAAGCTG
GACGCGACTTCTGAAGCTGTTGTCAAGGTGGATGTGGCTGAGGAGGAG
AAGGAGAAGAAGAAGAAGAAGAAGGGCACTGCGGAGGAGCCAGAGGAG
GAGGAGCCAGACGAGAGCATGCTGGACTGGTGGTCCAAGTACTTTGCC
TCCATTGACACCATGAAGGAGCAACTTCGACAACAAGAGCCCTCTGGA
ATTGACTTGGAGGAGAAGGAGGAAGTGGACAATACCGAGGGCCTGAAG
GGGTCAATGAAGGGCAAGGAGAAGGCAAGGGCTGCCAAAGAGGAGAAG
AAGAAGAAAACTCAGAGCTCTGGCTCTGGCCAGGGGTCCGAGGCCCCC
GAGAAGAAGAAACCCAAGATTGATGAGCTTAAGGTATACCCCAAAGAG
CTGGAGTCCGAGTTTGATAACTTTGAGGACTGGCTGCACACTTTCAAC
TTGCTTCGGGGCAAGACCGGGGATGATGAGGATGGCTCCACCGAGGAG
GAGCGCATTGTGGGACGCTTCAAGGGCTCCCTCTGCGTGTACAAAGTG
CCACTCCCAGAGGACGTGTCCCGGGAAGCCGGCTACGACTCCACCTAC
GGCATGTTCCAGGGCATCCCGAGCAATGACCCCATCAATGTGCTGGTC
CGAGTCTATGTGGTCCGGGCCACGGACCTGCACCCTGCTGACATCAAC
GGCAAAGCTGACCCCTACATCGCCATCCGGCTAGGCAAGACTGACATC
CGCGACAAGGAGAACTACATCTCCAAGCAGCTCAACCCTGTCTTTGGG
AAGTCCTTTGACATCGAGGCCTCCTTCCCCATGGAATCCATGCTGACG
GTGGCTGTGTATGACTGGGACCTGGTGGGCACTGATGACCTCATTGGG
GAAACCAAGATCGACCTGGAGAACCGCTTCTACAGCAAGCACCGCGCC
ACCTGCGGCATCGCCCAGACCTACTCCACACATGGCTACAATATCTGG
CGGGACCCCATGAAGCCCAGCCAGATCCTGACCCGCCTCTGCAAAGAC
GGCAAAGTGGACGGCCCCCACTTTGGGCCCCCTGGGAGAGTGAAGGTG
GCCAACCGCGTCTTCACTGGGCCCTCTGAGATTGAGGACGAGAACGGT
CAGAGGAAGCCCACAGACGAGCATGTGGCGCTGTTGGCCCTGAGGCAC
TGGGAGGACATCCCCCGCGCAGGCTGCCGCCTGGTGCCAGAGCATGTG
GAGACGAGGCCGCTGCTCAACCCCGACAAGCCGGGCATCGAGCAGGGC
CGCCTGGAGCTGTGGGTGGACATGTTCCCCATGGACATGCCAGCCCCT
GGGACGCCTCTGGACATCTCACCTCGGAAGCCCAAGAAGTACGAGCTG
CGGGTCATCATCTGGAACACAGATGAGGTGGTCTTGGAGGACGACGAC
TTCTTCACAGGGGAGAAGTCCAGTGACATCTTCGTGAGGGGGTGGCTG
AAGGGCCAGCAGGAGGACAAGCAGGACACAGACGTCCACTACCACTCC
CTCACTGGCGAGGGCAACTTCAACTGGCGCTACCTGTTCCCCTTCGAC
TACCTGGCGGCGGAGGAGAAGATCGTCATCTCCAAGAAGGAGTCCATG
TTCTCCTGGGACGAGACCGAGTACAAGATCCCCGCGCGGCTCACCCTG
CAGATCTGGGATGCGGACCACTTCTCCGCTGACGACTTCCTGGGGGCC
ATCGAGCTGGACCTGAACCGGTTCCCGCGGGGCGCAAAGACAGCCAAG
CAGTGCACCATGGAGATGGCCACCGGGGAGGTGGACGTGCCCCTCGTG
TCCATCTTCAAGCAAAAGCGCGTCAAAGGCTGGTGGCCCCTCCTGGCC
CGCAATGAGAACGATGAGTTTGAGCTCACGGGCAAGGTGGAGGCTGAG
CTGCATTTACTGACAGCAGAGGAGGCAGAGAAGAACCCAGTGGGCCTG
GCCCGCAATGAACCTGACCCCCTAGAGAAACCCAACCGGCCCGACACG
AGCTTCATCTGGTTCCTGAACCCTCTCAAGTCGGCTCGCTACTTCTTG
TGGCACACGTATCGCTGGCTGCTCCTCAAACTGTTGCTGCTCCTGCTG
CTGCTCCTCCTCCTCGCCCTGTTCCTCTACTCTGTGCCTGGCTACCTG
GTCAAGAAAATCCTCGGGGCCTGAGCGGCCGCGGTACCAAGGGCGAAT
TCTGCAGTCGACTAGAGCTCGCTGATCAGCCTCGACTGTGCCTTCTAG
TTGCCAGCCATCTGTTGTTTGCCCCTCCCCCGTGCCTTCCTTGACCCT
GGAAGGTGCCACTCCCACTGTCCTTTCCTAATAAAATGAGGAAATTGC
ATCGCATTGTCTGAGTAGGTGTCATTCTATTCTGGGGGGTGGGGTGGG
GCAGGACAGCAAGGGGGAGGATTGGGAAGACAATAGCAGGCATGCTGG
GGAGAGATCTGAGGACTAGTCCGTCGACTGTTAATTAAGCATGCTGGG
GAGAGATCTAGGAACCCCTAGTGATGGAGTTGGCCACTCCCTCTCTGC
GCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGG
GAGTGGCCAACCCCCCCCCCCCCCCCCCTGCAGCCCTGCATTAATGAA
TCGGCCAACGCGCGGGGAGAGGCGGTTTGCGTATTGGGCGCTCTTCCG
CTTCCTCGCTCACTGACTCGCTGCGCTCGGTCGTTCGGCTGCGGCGAG
CGGTATCAGCTCACTCAAAGGCGGTAATACGGTTATCCACAGAATCAG
GGGATAACGCAGGAAAGAACATGTGAGCAAAAGGCCAGCAAAAGGCCA
GGAACCGTAAAAAGGCCGCGTTGCTGGCGTTTTTCCATAGGCTCCGCC
CCCCTGACGAGCATCACAAAAATCGACGCTCAAGTCAGAGGTGGCGAA
ACCCGACAGGACTATAAAGATACCAGGCGTTTCCCCCTGGAAGCTCCC
TCGTGCGCTCTCCTGTTCCGACCCTGCCGCTTACCGGATACCTGTCCG
CCTTTCTCCCTTCGGGAAGCGTGGCGCTTTCTCAATGCTCACGCTGTA
GGTATCTCAGTTCGGTGTAGGTCGTTCGCTCCAAGCTGGGCTGTGTGC
ACGAACCCCCCGTTCAGCCCGACCGCTGCGCCTTATCCGGTAACTATC
GTCTTGAGTCCAACCCGGTAAGACACGACTTATCGCCACTGGCAGCAG
CCACTGGTAACAGGATTAGCAGAGCGAGGTATGTAGGCGGTGCTACAG
AGTTCTTGAAGTGGTGGCCTAACTACGGCTACACTAGAAGGACAGTAT
TTGGTATCTGCGCTCTGCTGAAGCCAGTTACCTTCGGAAAAAGAGTTG
GTAGCTCTTGATCCGGCAAACAAACCACCGCTGGTAGCGGTGGTTTTT
TTGTTTGCAAGCAGCAGATTACGCGCAGAAAAAAAGGATCTCAAGAAG
ATCCTTTGATCTTTTCTACGGGGTCTGACGCTCAGTGGAACGAAAACT
CACGTTAAGGGATTTTGGTCATGAGATTATCAAAAAGGATCTTCACCT
AGATCCTTTTAAATTAAAAATGAAGTTTTAAATCAATCTAAAGTATAT
ATGAGTAAACTTGGTCTGACAGTTACCAATGCTTAATCAGTGAGGCAC
CTATCTCAGCGATCTGTCTATTTCGTTCATCCATAGTTGCCTGACTCC
CCGTCGTGTAGATAACTACGATACGGGAGGGCTTACCATCTGGCCCCA
GTGCTGCAATGATACCGCGAGACCCACGCTCACCGGCTCCAGATTTAT
CAGCAATAAACCAGCCAGCCGGAAGGGCCGAGCGCAGAAGTGGTCCTG
CAACTTTATCCGCCTCCATCCAGTCTATTAATTGTTGCCGGGAAGCTA
GAGTAAGTAGTTCGCCAGTTAATAGTTTGCGCAACGTTGTTGCCATTG
CTACAGGCATCGTGGTGTCACGCTCGTCGTTTGGTATGGCTTCATTCA
GCTCCGGTTCCCAACGATCAAGGCGAGTTACATGATCCCCCATGTTGT
GCAAAAAAGCGGTTAGCTCCTTCGGTCCTCCGATCGTTGTCAGAAGTA
AGTTGGCCGCAGTGTTATCACTCATGGTTATGGCAGCACTGCATAATT
CTCTTACTGTCATGCCATCCGTAAGATGCTTTTCTGTGACTGGTGAGT
ACTCAACCAAGTCATTCTGAGAATAGTGTATGCGGCGACCGAGTTGCT
CTTGCCCGGCGTCAATACGGGATAATACCGCGCCACATAGCAGAACTT
TAAAAGTGCTCATCATTGGAAAACGTTCTTCGGGGCGAAAACTCTCAA
GGATCTTACCGCTGTTGAGATCCAGTTCGATGTAACCCACTCGTGCAC
CCAACTGATCTTCAGCATCTTTTACTTTCACCAGCGTTTCTGGGTGAG
CAAAAACAGGAAGGCAAAATGCCGCAAAAAAGGGAATAAGGGCGACAC
GGAAATGTTGAATACTCATACTCTTCCTTTTTCAATATTATTGAAGCA
TTTATCAGGGTTATTGTCTCATGAGCGGATACATATTTGAATGTATTT
AGAAAAATAAACAAATAGGGGTTCCGCGCACATTTCCCCGAAAAGTGC
CACCTGACGTCTAAGAAACCATTATTATCATGACATTAACCTATAAAA
ATAGGCGTATCACGAGGCCCTTTCGTCTCGCGCGTTTCGGTGATGACG
GTGAAAACCTCTGACACATGCAGCTCCCGGAGACGGTCACAGCTTGTC
TGTAAGCGGATGCCGGGAGCAGACAAGCCCGTCAGGGCGCGTCAGCGG
GTGTTGGCGGGTGTCGGGGCTGGCTTAACTATGCGGCATCAGAGCAGA
TTGTACTGAGAGTGCACCATATGCGGTGTGAAATACCGCACAGATGCG
TAAGGAGAAAATACCGCATCAGGAAATTGTAAACGTTAATATTTTGTT
AAAATTCGCGTTAAATTTTTGTTAAATCAGCTCATTTTTTAACCAATA
GGCCGAAATCGGCAAAATCCCTTATAAATCAAAAGAATAGACCGAGAT
AGGGTTGAGTGTTGTTCCAGTTTGGAACAAGAGTCCACTATTAAAGAA
CGTGGACTCCAACGTCAAAGGGCGAAAAACCGTCTATCAGGGCGATGG
CCCACTACGTGAACCATCACCCTAATCAAGTTTTTTGGGGTCGAGGTG
CCGTAAAGCACTAAATCGGAACCCTAAAGGGAGCCCCCGATTTAGAGC
TTGACGGGGAAAGCCGGCGAACGTGGCGAGAAAGGAAGGGAAGAAAGC
GAAAGGAGCGGGCGCTAGGGCGCTGGCAAGTGTAGCGGTCACGCTGCG
CGTAACCACCACACCCGCCGCGCTTAATGCGCCGCTACAGGGCGCGTC
GCGCCATTCGCCATTCAGGCTACGCAACTGTTGGGAAGGGCGATCGGT
GCGGGCCTCTTCGCTATTACGCCAGGCTGC
pTR22-APhead-APSA-otoferlinCT Hs var 5
(SEQ ID NO: 16)
AGGGGGGGGGGGGGGGGGGTTGGCCACTCCCTCTCTGCGCGCTCGCTC
GCTCACTGAGGCCGGGCGACCAAAGGTCGCCCGACGCCCGGGCTTTGC
CCGGGCGGCCTCAGTGAGCGAGCGAGCGCGCAGAGAGGGAGTGGCCAA
CTCCATCACTAGGGGTTCCTCAGATCTGGCGCGCCCAATTGGCTTCGA
ATTCTAGCGGCCGCCCCCGGGTGCGCGGCGTCGGTGGTGCCGGCGGGG
GGCGCCAGGTCGCAGGCGGTGTAGGGCTCCAGGCAGGCGGCGAAGGCC
ATGACGTGCGCTATGAAGGTCTGCTCCTGCACGCCGTGAACCAGGTGC
GCCTGCGGGCCGCGCGCGAACACCGCCACGTCCTCGCCTGCGTGGGTC
TCTTCGTCCAGGGGCACTGCTGACTGCTGCCGATACTCGGGGCTCCCG
CTCTCGCTCTCGGTAACATCCGGCCGGGCGCCGTCCTTGAGCACATAG
CCTGGACCGTTTCCTTAAGCGACGCATGCTCGCGATAGGCACCTATTG
GTCTTACTGACATCCACTTTGCCTTTCTCTCCACAGGAAAACATGGGG
CAGCAGGCCAGGATGCTGCGGGCCCAGGTGAAGCGGCACACGGTGCGG
GACAAGCTGAGGCTGTGCCAGAACTTCCTGCAGAAGCTGCGCTTCCTG
GCGGACGAGCCCCAGCACAGCATTCCCGACATCTTCATCTGGATGATG
AGCAACAACAAGCGTGTCGCCTATGCCCGTGTGCCCTCCAAGGACCTG
CTCTTCTCCATCGTGGAGGAGGAGACTGGCAAGGACTGCGCCAAGGTC
AAGACGCTCTTCCTTAAGCTGCCAGGGAAGCGGGGCTTCGGCTCGGCA
GGCTGGACAGTGCAGGCCAAGGTGGAGCTGTACCTGTGGCTGGGCCTC
AGCAAACAGCGCAAGGAGTTCCTGTGCGGCCTGCCCTGTGGCTTCCAG
GAGGTCAAGGCAGCCCAGGGCCTGGGCCTGCATGCCTTCCCACCCGTC
AGCCTGGTCTACACCAAGAAGCAGGCGTTCCAGCTCCGAGCGCACATG
TACCAGGCCCGCAGCCTCTTTGCCGCCGACAGCAGCGGACTCTCAGAC
CCCTTTGCCCGCGTCTTCTTCATCAATCAGAGTCAGTGCACAGAGGTG
CTGAATGAGACCCTGTGTCCCACCTGGGACCAGATGCTGGTGTTCGAC
AACCTGGAGCTCTATGGTGAAGCTCATGAGCTGAGGGACGATCCGCCC
ATCATTGTCATTGAAATCTATGACCAGGATTCCATGGGCAAAGCTGAC
TTCATGGGCCGGACCTTCGCCAAACCCCTGGTGAAGATGGCAGACGAG
GCGTACTGCCCACCCCGCTTCCCACCTCAGCTCGAGTACTACCAGATC
TACCGTGGCAACGCCACAGCTGGAGACCTGCTGGCGGCCTTCGAGCTG
CTGCAGATTGGACCAGCAGGGAAGGCTGACCTGCCCCCCATCAATGGC
CCGGTGGACGTGGACCGAGGTCCCATCATGCCCGTGCCCATGGGCATC
CGGCCCGTGCTCAGCAAGTACCGAGTGGAGGTGCTGTTCTGGGGCCTA
CGGGACCTAAAGCGGGTGAACCTGGCCCAGGTGGACCGGCCACGGGTG
GACATCGAGTGTGCAGGGAAGGGGGTGCAGTCGTCCCTGATCCACAAT
TATAAGAAGAACCCCAACTTCAACACCCTCGTCAAGTGGTTTGAAGTG
GACCTCCCAGAGAACGAGCTGCTGCACCCGCCCTTGAACATCCGTGTG
GTGGACTGCCGGGCCTTCGGTCGCTACACACTGGTGGGCTCCCATGCC
GTCAGCTCCCTGCGACGCTTCATCTACCGGCCCCCAGACCGCTCGGCC
CCCAGCTGGAACACCACGGTCAGGCTTCTCCGGCGCTGCCGTGTGCTG
TGCAATGGGGGCTCCTCCTCTCACTCCACAGGGGAGGTTGTGGTGACT
ATGGAGCCAGAGGTACCCATCAAGAAACTGGAGACCATGGTGAAGCTG
GACGCGACTTCTGAAGCTGTTGTCAAGGTGGATGTGGCTGAGGAGGAG
AAGGAGAAGAAGAAGAAGAAGAAGGGCACTGCGGAGGAGCCAGAGGAG
GAGGAGCCAGACGAGAGCATGCTGGACTGGTGGTCCAAGTACTTTGCC
TCCATTGACACCATGAAGGAGCAACTTCGACAACAAGAGCCCTCTGGA
ATTGACTTGGAGGAGAAGGAGGAAGTGGACAATACCGAGGGCCTGAAG
GGGTCAATGAAGGGCAAGGAGAAGGCAAGGGCTGCCAAAGAGGAGAAG
AAGAAGAAAACTCAGAGCTCTGGCTCTGGCCAGGGGTCCGAGGCCCCC
GAGAAGAAGAAACCCAAGATTGATGAGCTTAAGGTATACCCCAAAGAG
CTGGAGTCCGAGTTTGATAACTTTGAGGACTGGCTGCACACTTTCAAC
TTGCTTCGGGGCAAGACCGGGGATGATGAGGATGGCTCCACCGAGGAG
GAGCGCATTGTGGGACGCTTCAAGGGCTCCCTCTGCGTGTACAAAGTG
CCACTCCCAGAGGACGTGTCCCGGGAAGCCGGCTACGACTCCACCTAC
GGCATGTTCCAGGGCATCCCGAGCAATGACCCCATCAATGTGCTGGTC
CGAGTCTATGTGGTCCGGGCCACGGACCTGCACCCTGCTGACATCAAC
GGCAAAGCTGACCCCTACATCGCCATCCGGCTAGGCAAGACTGACATC
CGCGACAAGGAGAACTACATCTCCAAGCAGCTCAACCCTGTCTTTGGG
AAGTCCTTTGACATCGAGGCCTCCTTCCCCATGGAATCCATGCTGACG
GTGGCTGTGTATGACTGGGACCTGGTGGGCACTGATGACCTCATTGGG
GAAACCAAGATCGACCTGGAGAACCGCTTCTACAGCAAGCACCGCGCC
ACCTGCGGCATCGCCCAGACCTACTCCACACATGGCTACAATATCTGG
CGGGACCCCATGAAGCCCAGCCAGATCCTGACCCGCCTCTGCAAAGAC
GGCAAAGTGGACGGCCCCCACTTTGGGCCCCCTGGGAGAGTGAAGGTG
GCCAACCGCGTCTTCACTGGGCCCTCTGAGATTGAGGACGAGAACGGT
CAGAGGAAGCCCACAGACGAGCATGTGGCGCTGTTGGCCCTGAGGCAC
TGGGAGGACATCCCCCGCGCAGGCTGCCGCCTGGTGCCAGAGCATGTG
GAGACGAGGCCGCTGCTCAACCCCGACAAGCCGGGCATCGAGCAGGGC
CGCCTGGAGCTGTGGGTGGACATGTTCCCCATGGACATGCCAGCCCCT
GGGACGCCTCTGGACATCTCACCTCGGAAGCCCAAGAAGTACGAGCTG
CGGGTCATCATCTGGAACACAGATGAGGTGGTCTTGGAGGACGACGAC
TTCTTCACAGGGGAGAAGTCCAGTGACATCTTCGTGAGGGGGTGGCTG
AAGGGCCAGCAGGAGGACAAGCAGGACACAGACGTCCACTACCACTCC
CTCACTGGCGAGGGCAACTTCAACTGGCGCTACCTGTTCCCCTTCGAC
TACCTGGCGGCGGAGGAGAAGATCGTCATCTCCAAGAAGGAGTCCATG
TTCTCCTGGGACGAGACCGAGTACAAGATCCCCGCGCGGCTCACCCTG
CAGATCTGGGATGCGGACCACTTCTCCGCTGACGACTTCCTGGGGGCC
ATCGAGCTGGACCTGAACCGGTTCCCGCGGGGCGCAAAGACAGCCAAG
CAGTGCACCATGGAGATGGCCACCGGGGAGGTGGACGTGCCCCTCGTG
TCCATCTTCAAGCAAAAGCGCGTCAAAGGCTGGTGGCCCCTCCTGGCC
CGCAATGAGAACGATGAGTTTGAGCTCACGGGCAAGGTGGAGGCTGAG
CTGCATTTACTGACAGCAGAGGAGGCAGAGAAGAACCCAGTGGGCCTG
GCCCGCAATGAACCTGACCCCCTAGAGAAACCCAACCGGCCCGACACG
GCCTTCGTCTGGTTCCTCAACCCTCTCAAGTCCATCAAGTACCTCATC
TGCACCCGGTACAAGTGGCTCATCATCAAGATCGTGCTGGCGCTGTTG
GGGCTGCTCATGTTGGGGCTCTTCCTCTACAGCCTCCCTGGCTACATG
GTCAAAAAGCTCCTTGGGGCATGAGCGGCCGCGGTACCAAGGGCGAAT
TCTGCAGTCGACTAGAGCTCGCTGATCAGCCTCGACTGTGCCTTCTAG
TTGCCAGCCATCTGTTGTTTGCCCCTCCCCCGTGCCTTCCTTGACCCT
GGAAGGTGCCACTCCCACTGTCCTTTCCTAATAAAATGAGGAAATTGC
ATCGCATTGTCTGAGTAGGTGTCATTCTATTCTGGGGGGTGGGGTGGG
GCAGGACAGCAAGGGGGAGGATTGGGAAGACAATAGCAGGCATGCTGG
GGAGAGATCTGAGGACTAGTCCGTCGACTGTTAATTAAGCATGCTGGG
GAGAGATCTAGGAACCCCTAGTGATGGAGTTGGCCACTCCCTCTCTGC
GCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGG
GAGTGGCCAACCCCCCCCCCCCCCCCCCTGCAGCCCTGCATTAATGAA
TCGGCCAACGCGCGGGGAGAGGCGGTTTGCGTATTGGGCGCTCTTCCG
CTTCCTCGCTCACTGACTCGCTGCGCTCGGTCGTTCGGCTGCGGCGAG
CGGTATCAGCTCACTCAAAGGCGGTAATACGGTTATCCACAGAATCAG
GGGATAACGCAGGAAAGAACATGTGAGCAAAAGGCCAGCAAAAGGCCA
GGAACCGTAAAAAGGCCGCGTTGCTGGCGTTTTTCCATAGGCTCCGCC
CCCCTGACGAGCATCACAAAAATCGACGCTCAAGTCAGAGGTGGCGAA
ACCCGACAGGACTATAAAGATACCAGGCGTTTCCCCCTGGAAGCTCCC
TCGTGCGCTCTCCTGTTCCGACCCTGCCGCTTACCGGATACCTGTCCG
CCTTTCTCCCTTCGGGAAGCGTGGCGCTTTCTCAATGCTCACGCTGTA
GGTATCTCAGTTCGGTGTAGGTCGTTCGCTCCAAGCTGGGCTGTGTGC
ACGAACCCCCCGTTCAGCCCGACCGCTGCGCCTTATCCGGTAACTATC
GTCTTGAGTCCAACCCGGTAAGACACGACTTATCGCCACTGGCAGCAG
CCACTGGTAACAGGATTAGCAGAGCGAGGTATGTAGGCGGTGCTACAG
AGTTCTTGAAGTGGTGGCCTAACTACGGCTACACTAGAAGGACAGTAT
TTGGTATCTGCGCTCTGCTGAAGCCAGTTACCTTCGGAAAAAGAGTTG
GTAGCTCTTGATCCGGCAAACAAACCACCGCTGGTAGCGGTGGTTTTT
TTGTTTGCAAGCAGCAGATTACGCGCAGAAAAAAAGGATCTCAAGAAG
ATCCTTTGATCTTTTCTACGGGGTCTGACGCTCAGTGGAACGAAAACT
CACGTTAAGGGATTTTGGTCATGAGATTATCAAAAAGGATCTTCACCT
AGATCCTTTTAAATTAAAAATGAAGTTTTAAATCAATCTAAAGTATAT
ATGAGTAAACTTGGTCTGACAGTTACCAATGCTTAATCAGTGAGGCAC
CTATCTCAGCGATCTGTCTATTTCGTTCATCCATAGTTGCCTGACTCC
CCGTCGTGTAGATAACTACGATACGGGAGGGCTTACCATCTGGCCCCA
GTGCTGCAATGATACCGCGAGACCCACGCTCACCGGCTCCAGATTTAT
CAGCAATAAACCAGCCAGCCGGAAGGGCCGAGCGCAGAAGTGGTCCTG
CAACTTTATCCGCCTCCATCCAGTCTATTAATTGTTGCCGGGAAGCTA
GAGTAAGTAGTTCGCCAGTTAATAGTTTGCGCAACGTTGTTGCCATTG
CTACAGGCATCGTGGTGTCACGCTCGTCGTTTGGTATGGCTTCATTCA
GCTCCGGTTCCCAACGATCAAGGCGAGTTACATGATCCCCCATGTTGT
GCAAAAAAGCGGTTAGCTCCTTCGGTCCTCCGATCGTTGTCAGAAGTA
AGTTGGCCGCAGTGTTATCACTCATGGTTATGGCAGCACTGCATAATT
CTCTTACTGTCATGCCATCCGTAAGATGCTTTTCTGTGACTGGTGAGT
ACTCAACCAAGTCATTCTGAGAATAGTGTATGCGGCGACCGAGTTGCT
CTTGCCCGGCGTCAATACGGGATAATACCGCGCCACATAGCAGAACTT
TAAAAGTGCTCATCATTGGAAAACGTTCTTCGGGGCGAAAACTCTCAA
GGATCTTACCGCTGTTGAGATCCAGTTCGATGTAACCCACTCGTGCAC
CCAACTGATCTTCAGCATCTTTTACTTTCACCAGCGTTTCTGGGTGAG
CAAAAACAGGAAGGCAAAATGCCGCAAAAAAGGGAATAAGGGCGACAC
GGAAATGTTGAATACTCATACTCTTCCTTTTTCAATATTATTGAAGCA
TTTATCAGGGTTATTGTCTCATGAGCGGATACATATTTGAATGTATTT
AGAAAAATAAACAAATAGGGGTTCCGCGCACATTTCCCCGAAAAGTGC
CACCTGACGTCTAAGAAACCATTATTATCATGACATTAACCTATAAAA
ATAGGCGTATCACGAGGCCCTTTCGTCTCGCGCGTTTCGGTGATGACG
GTGAAAACCTCTGACACATGCAGCTCCCGGAGACGGTCACAGCTTGTC
TGTAAGCGGATGCCGGGAGCAGACAAGCCCGTCAGGGCGCGTCAGCGG
GTGTTGGCGGGTGTCGGGGCTGGCTTAACTATGCGGCATCAGAGCAGA
TTGTACTGAGAGTGCACCATATGCGGTGTGAAATACCGCACAGATGCG
TAAGGAGAAAATACCGCATCAGGAAATTGTAAACGTTAATATTTTGTT
AAAATTCGCGTTAAATTTTTGTTAAATCAGCTCATTTTTTAACCAATA
GGCCGAAATCGGCAAAATCCCTTATAAATCAAAAGAATAGACCGAGAT
AGGGTTGAGTGTTGTTCCAGTTTGGAACAAGAGTCCACTATTAAAGAA
CGTGGACTCCAACGTCAAAGGGCGAAAAACCGTCTATCAGGGCGATGG
CCCACTACGTGAACCATCACCCTAATCAAGTTTTTTGGGGTCGAGGTG
CCGTAAAGCACTAAATCGGAACCCTAAAGGGAGCCCCCGATTTAGAGC
TTGACGGGGAAAGCCGGCGAACGTGGCGAGAAAGGAAGGGAAGAAAGC
GAAAGGAGCGGGCGCTAGGGCGCTGGCAAGTGTAGCGGTCACGCTGCG
CGTAACCACCACACCCGCCGCGCTTAATGCGCCGCTACAGGGCGCGTC
GCGCCATTCGCCATTCAGGCTACGCAACTGTTGGGAAGGGCGATCGGT
GCGGGCCTCTTCGCTATTACGCCAGGCTGC

### REFERENCES

1) Adato Al, Raskin L, Petit C, Bonne-Tamir B Deafness heterogeneity in a Druze isolate from the Middle East: novel OTOF and PDS mutations, low prevalence of GJB2 35delG mutation and indication for a new DFNB locus.Eur J Hum Genet. 2000 Jun;8(6):437-42.
2) Allocca M, Doria M, Petrillo M, Colella P, Garcia-Hoyos M, Gibbs D, Kim SR, Maguire A, Rex TS, Di Vicino U, Cutillo L, Sparrow JR, Williams DS, Bennett J, Auricchio A. Serotype-dependent packaging of large genes in adeno-associated viral vectors results in effective gene delivery in mice. J Clin Invest. 2008 May;118(5):1955-64.
3) Chaib, H., Place, C., Salem, N., Chardenoux, S., Vincent, C., Weissenbach, J., El-Zir, E., Loiselet, J., Petit, C. A gene responsible for a sensorineural nonsyndromic recessive deafness maps to chromosome 2p22-23. Hum. Molec. Genet. 1996 5: 155-158.
4) Choi, B. Y., Ahmed, Z. M., Riazuddin, S., Bhinder, M. A., Shahzad, M., Husnain, T., Riazuddin, S., Griffith, A. J., Friedman, T. B. Identities and frequencies of mutations of the otoferlin gene (OTOF) causing DFNB9 deafness in Pakistan. Clin. Genet. 2009 75:237-243.
5) Dong B, Nakai H, Xiao W. Characterization of genome integrity for oversized recombinant AAV vector. Mol Ther. 2010 Jan;18(1):87-92.
6) Ghosh A, Yue Y, Duan D. Efficient transgene reconstitution with hybrid dual AAV vectors carrying the minimized bridging sequences. Hum Gene Ther. 2011 Jan;22(1):77-83.
7) Hirsch ML, Agbandje-McKenna M, Samulski RJ. Little vector, big gene transduction: fragmented genome reassembly of adeno-associated virus. Mol Ther. 2010 Jan;18(1):6-8.
8) Lai Y, Yue Y, Duan D. Evidence for the failure of adeno-associated virus serotype 5 to package a viral genome> or =8.2 kb. Mol Ther. 2010 Jan;18(1):75-9.
9) Matsunaga Tl, Mutai H, Kunishima S, Namba K, Morimoto N, Shinjo Y, Arimoto Y, Kataoka Y, Shintani T, Morita N, Sugiuchi T, Masuda S, Nakano A, Taiji H, Kaga K. A prevalent founder mutation and genotype-phenotype correlations of OTOF in Japanese patients with auditory neuropathy. Clin Genet. 2012 Nov;82(5):425-32. doi: 10.1111/j.1399-0004.2012.01897.x. Epub 2012 Jun 1.
10) Rodríguez-Ballesteros M, del Castillo FJ, Martín Y, Moreno-Pelayo MA, Morera C, Prieto F, Marco J, Morant A, Gallo-Terán J, Morales-Angulo C, Navas C, Trinidad G, Tapia MC, Moreno F, del Castillo I. Auditory neuropathy in patients carrying mutations in the otoferlin gene (OTOF). Hum Mutat. 2003 Dec;22(6):451-6.
11) Roux I, Safieddine S, Nouvian R, Grati M, Simmler MC, Bahloul A, Perfettini I, Le Gall M, Rostaing P, Hamard G, Triller A, Avan P, Moser T, Petit C. Otoferlin, defective in a human deafness form, is essential for exocytosis at the auditory ribbon synapse. Cell. 2006 Oct 20;127(2):277-89.
12) Wu Z, Yang H, Colosi P. Effect of genome size on AAV vector packaging. Mol Ther. 2010 Jan;18(1):80-6.
13) Yasunaga S, Grati M, Chardenoux S, Smith TN, Friedman TB, Lalwani AK, Wilcox ER, Petit C. Am J Hum Genet. OTOF encodes multiple long and short isoforms: genetic evidence that the long ones underlie recessive deafness DFNB9. 2000 Sep;67(3):591-600. Epub 2000 Jul 19.
14) Yasunaga S, Grati M, Cohen-Salmon M, El-Amraoui A, Mustapha M, Salem N, El-Zir E, Loiselet J, Petit C. A mutation in OTOF, encoding otoferlin, a FER-1-like protein, causes DFNB9, a nonsyndromic form of deafness. Nat Genet. 1999 Apr;21(4):363-9.
15) Didier Dulon, Saaid Safieddine, Sherri M. Jones, Christine Petit. Otoferlin is Critical for a Highly Sensitive and Linear Calcium Dependent Exocytosis at Vestibular Hair Cell Ribbon Synapses. J Neurosci. 2009 August.
16) Zippora Brownstein, Yoni Bhonker and Karen B Avraham. High-throughput sequencing to decipher the genetic heterogeneity of deafness. Brownstein et al. Genome Biology 2012, 13:245
17) Rodríguez-Ballesteros et al. (2003) “Auditory neuropathy in patients carrying mutations in the otoferlin gene (OTOF)” Hum Mutat.; 22 (6):451-456.
18) Petersen MB, Willems PJ: Non-syndromic, autosomal-recessive deafness. Clin Genet. 2006; 69 (5): 371-92.
19) Smith R, Gurrola J, Kelley P. OTOF-Related Deafness. In: Pagon R, Bird T, Dolan C, Stephens K, eds. Gene Reviews. Seattle: Internet; 2008
20) Roux I, Safieddine S, Nouvian R et al. Otoferlin, defective in a human deafness form, is essential for exocytosis at the auditory ribbon synapse. Cell 2006;127:277-89
21) Kral A, O'Donoghue GM: Profound deafness in childhood. N Engl J Med. 2010; 363(15):1438-50. doi: 10.1056/NEJMra0911225.
22) Dyka FM, Boye SL, Chiodo VA, Hauswirth WW, Boye SE., Dual Adeno-Associated Virus Vectors Result in Efficient In Vitro and In Vivo Expression of an Oversized Gene MY07A, Hum Gene Ther Methods. 2014; 25 (2):166-77. doi: 10.1089/hgtb.2013.212.
23) Akil O, Seal RP, Burke K, Wang C, Alemi A, During M, Edwards RH, Lustig LR: Restoration of hearing in the VGLUT3 knockout mouse using virally mediated gene therapy. Neuron. 2012; 75 (2): 283-93. doi: 10.1016/j.neuron.2012.05.019.

### OTHER EMBODIMENTS

All of the features disclosed in this specification may be combined in any combination.

Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1.-28. (canceled)

29. A composition comprising:
a first AAV particle comprising a first polynucleotide; and
a second AAV particle comprising a second polynucleotide, wherein
(i) the first polynucleotide comprises inverted terminal repeat (ITR) sequences flanking a first expression cassette containing, from 5′ to 3′:
(a) a promoter,
(b) a partial coding sequence that encodes an N-terminal portion of an Otoferlin polypeptide,
(c) a splice donor site, and
(d) a first region of homology containing a sequence that is homologous to a sequence in the second polynucleotide, and
(ii) the second polynucleotide comprises ITR sequences flanking a second expression cassette containing, from 5′ to 3′:
(a) a second region of homology containing a sequence that is homologous to a sequence in the first polynucleotide,
(b) a splice acceptor site,
(c) a partial coding sequence that encodes a C-terminal portion of the Otoferlin polypeptide, and
(d) a polyadenylation (pA) signal sequence,
wherein the Otoferlin polypeptide is a human Otoferlin isoform 5 polypeptide.

30. The composition of claim 29, wherein the first region of homology and the second region of homology are between 50 and 500 nucleotides.

31. The composition of claim 30, wherein the first region of homology and the second region of homology are between 50 and 300 nucleotides.

32. The composition of claim 31, wherein each of the first region of homology and the second region of homology comprises the nucleotide sequence of SEQ ID NO: 3.

33. The composition of claim 29, wherein the promoter is a chimeric cytomegalovirus (CMV)/chicken β actin promoter or a truncated chimeric CMV/chicken β actin promoter.

34. The composition of claim 33, wherein the promoter comprises the nucleotide sequence of SEQ ID NO: 4.

35. The composition of claim 29, wherein the Otoferlin polypeptide comprises the amino acid sequence of SEQ ID NO: 6.

36. The composition of claim 29, wherein the splice donor site comprises the nucleotide sequence of SEQ ID NO: 7.

37. The composition of claim 29, wherein the splice acceptor site comprises the nucleotide sequence of SEQ ID NO: 8.

38. The composition of claim 29, wherein the ITR sequences are AAV2 ITR sequences.

39. The composition of claim 29, wherein the first and second AAV particles are AAV2 serotype particles.

40. The composition of claim 29, wherein the first and second AAV particles are AAV1 serotype particles.

41. The composition of claim 29, further comprising a pharmaceutically acceptable carrier.
